(19) United States
(12) Patent Application Publication
(10) Pub. No.: US 2012/0329046 A1
(43) Pub. Date: Dec. 27, 2012

(54) MOLECULAR MARKER FOR EVALUATING PATHOLOGICAL CONDITIONS AND TREATMENT OF MUSCULAR DYSTROPHY

(75) Inventors: Shin'ichi Takeda, Tokyo (JP); Akinori Nakamura, Tokyo (JP); Masanori Kobayashi, Tokyo (JP); Takashi Okada, Tokyo (JP)

(73) Assignee: National Center of Neurology and Psychiatry, Tokyo (JP)

(21) Appl. No.: 13/461,075

(22) Filed: May 1, 2012

(30) Foreign Application Priority Data
Jun. 27, 2011 (JP) 2011-142312

(51) Int. Cl. G01N 33/566; C12Q 1/68

(52) U.S. Cl. 435/6.11; 435/6.12; 435/7.9; 435/7.21; 436/501; 435/7.1; 435/6.13

(57) ABSTRACT

Novel markers associated with the development of muscular dystrophy that elucidate the mechanisms of muscular dystrophy development and provide a means for diagnosis and treatment of muscular dystrophy are presented. The expression level of one or more markers selected from the group consisting of c-Fos, EGR1, IL-6, and IL-8 in a sample obtained from the subject can be compared with a reference value to diagnose muscular dystrophy in the subject.

### CROSS-REFERENCE TO RELATED APPLICATIONS

The present application claims priority from Japanese patent application JP 2011-142312 filed on Jun. 27, 2011, the content of which is hereby incorporated by reference into this application.

### FIELD OF THE INVENTION

The present invention relates to a novel marker for muscular dystrophy. More specifically, the present invention relates to a diagnostic method for muscular dystrophy. In addition, the present invention relates to a method for screening for a therapeutic agent or technique for muscular dystrophy and a method for evaluating efficacy of a therapeutic agent or technique for muscular dystrophy.

### BACKGROUND OF THE INVENTION

“Muscular dystrophy” is a generic name for hereditary diseases causing progressive amyotrophia and muscular weakness throughout the body. Among various types of muscular dystrophy, Duchenne muscular dystrophy (DMD) is associated with X chromosome, and is the most frequent type (i.e., 1 patient per 3,500 newborn males). In general, DMD is a very serious disease, which develops as gait disturbance at the age of 2 to 5 and advances to gait inability up to the age of 13, and patients die of respiratory failure or cardiac failure at around age 30. DMD develops due to mutation of the dystrophin gene that encodes dystrophin distributed in the sarcolemma. While dystrophin is believed to have functions of stabilizing muscular fibers and maintaining intracellular calcium homeostasis between muscle contraction and muscle relaxation (Infante, J. P., et al., Mol. Cell. Biochem., 1999, 195: 155-167), it is considered that dystrophin deficiency results in a weakened sarcolemma and elevated calcium level in the muscle cells, which lead to activation of various proteases or myonecrosis (Hopf, F. W., et al., Am. J. Physiol., 1996, 271: C1325-C1339). It is important to understand the pathological mechanisms of muscular dystrophy in order to develop therapeutic techniques. To this end, animal models are essential. However, the mdx mice, which are the most frequently employed DMD animal models, exhibit very active muscle regeneration in addition to myonecrosis (Tanabe, Y., et al., Acta Neuropathologica (Berl) 1986, 69: 91-95), and the molecular mechanisms of myodystrophy have not yet been fully elucidated.

It has heretofore been reported that the serum or plasma creatine kinase (CK) level has been high in the case of neonatal DMD (Heyck, H. et al., Klin. Wescher, 1966, 44 695-700; Demos, J., Am. J. Phys. Med., 1971, 50: 271-284; Zellweger, H. et al., Pediatrics, 1975, 55: 3-4; and Ionasescu, V. et al., Lancet, 1978, 2: 1251). However, causes thereof have not yet been fully elucidated, and techniques for neonatal diagnosis of DMD have not yet been established. Meanwhile, other animal models of DMD (i.e., dog models of muscular dystrophy; they may be referred to as “dystrophic dogs”) exhibit significantly high serum CK levels at birth, and the fatality rate at the newborn stage is also high (Valentine, B. A. et al., J. Neurol. Sci., 1988, 88: 69-81; and Shimatsu, Y. et al., Acta Myologica, 2005, 24: 145-154), although detailed causes thereof remain unknown.

### SUMMARY OF THE INVENTION

Diagnosis of muscular dystrophy has involved the use of serum CK levels since the serum CK levels are significantly elevated by myopathy or necrosis. Since the serum CK levels are easily changed with motion or at rest, it has been pointed out that such levels are insufficient for diagnosis or comprehension of disease progression.

Accordingly, objects of the present invention are provision of a novel marker associated with the development of muscular dystrophy, elucidation of the developmental mechanisms of muscular dystrophy, and provision of means for diagnosis and treatment of muscular dystrophy.

The present inventors have conducted concentrated studies in order to attain the above objects. As a result, we discovered four novel markers associated with muscular dystrophy (i.e., c-Fos, EGR1, IL-6, and IL-8), and found that the utilization of the expression levels of such markers would enable diagnosis of muscular dystrophy or screening for a therapeutic agent or technique for muscular dystrophy. This has led to the completion of the present invention.

Specifically, the present invention encompasses [1] to [11] below.

[1] A diagnostic agent for muscular dystrophy comprising a means for determining expression level of at least one marker selected from the group consisting of c-Fos, EGR1, IL-6, and IL-8 in a sample.

[2] The diagnostic agent according to [1], wherein the means is a DNA primer and/or DNA probe.

[3] The diagnostic agent according to [1], wherein the means is an antibody.

[4] The diagnostic agent according to any of [1] to [3], wherein the sample is selected from the group consisting of a muscle sample, a blood sample, and a serum sample.

[5] The diagnostic agent according to any of [1] to [4], wherein muscular dystrophy diagnosis is evaluation of carrying of muscular dystrophy or prediction of development of muscular dystrophy.

[6] A method of diagnosing muscular dystrophy in a subject comprising:

(a) determining expression level of at least one marker selected from the group consisting of c-Fos, EGR1, IL-6, and IL-8 in a sample obtained from the subject; and

(b) diagnosing muscular dystrophy for the subject by comparing the results of step (a) with a reference.

[7] A method for screening for a therapeutic agent or technique for muscular dystrophy comprising:

(a) treating a muscle cell derived from an animal that had developed muscular dystrophy or a muscular dystrophy carrier animal with a test agent or technique;

(b) determining expression level of at least one marker selected from the group consisting of c-Fos, EGR1, IL-6, and IL-8 in the muscle cell; and (c) identifying a test agent or technique as a candidate for the therapeutic agent or technique for muscular dystrophy based on the results obtained in step (b).

[8] A method for screening for a therapeutic agent or technique for muscular dystrophy comprising:

(a) determining expression level of at least one marker selected from the group consisting of c-Fos, EGR1, IL-6, and IL-8 in a sample obtained from an animal that had developed muscular dystrophy or a muscular dystrophy carrier animal, which has been treated with a test agent or technique; and

(b) identifying a test agent or technique as a candidate for the therapeutic agent or technique for muscular dystrophy based on the results obtained in step (a).

[9] The method according to [7] or [8], which further comprises a step of determining expression level of at least one marker selected from the group consisting of c-Fos, EGR1, IL-6, and IL-8 in the muscle cell or sample prior to the treatment with the test agent or technique.

[10] The method according to any of [7] to [9], wherein the test agent or technique is identified as a candidate for the therapeutic agent or technique for muscular dystrophy when the expression level of a marker in the muscle cell or sample is lower than that of the same marker in an untreated muscle cell or sample.

[11] A method for evaluating the efficacy of a therapeutic agent or technique for muscular dystrophy comprising:

(a) determining expression level of at least one marker selected from the group consisting of c-Fos, EGR1, IL-6, and IL-8 in a sample obtained from an animal that had developed muscular dystrophy or a muscular dystrophy carrier animal, which has been treated with a test agent or technique; and

(b) evaluating the efficacy of the test agent or technique based on the results obtained in step (a).

[12] The method according to any of [7] to [11], wherein the animal that had developed muscular dystrophy or the muscular dystrophy carrier animal is a human who had developed muscular dystrophy or is a muscular dystrophy carrier, or an animal model of muscular dystrophy.

[13] A therapeutic or preventive agent for muscular dystrophy comprising a means for inhibiting or suppressing expression or activity of at least one marker selected from the group consisting of c-Fos, EGR1, IL-6, and IL-8.

The diagnostic method for muscular dystrophy according to the present invention enables early diagnosis and prediction of carrying and future development of muscular dystrophy, and it is thus useful for early treatment of muscular dystrophy. In addition, the novel marker according to the present invention is associated with muscular dystrophy, and it can be used for elucidation of the developmental mechanisms of muscular dystrophy and development of therapeutic techniques or agents for the same, in addition to diagnosis and prediction of muscular dystrophy.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows charts showing postnatal creatinine kinase (CK) levels in normal dogs, carrier dogs, and dystrophic dogs. (A) shows postnatal CK levels in newborn dogs delivered via spontaneous labor (SL) and caesarean section (CS); (B) shows serum CK levels in the umbilical cord (UC) blood and in the venous blood of newborn dogs after the initiation of breathing; and (C) shows changes in serum CK levels of newborn dogs over time (from the serum CK levels prior to the initiation of breathing to those 48 hours after birth).

FIG. 2 shows photographs showing histopathological changes of the diaphragm of newborn dystrophic dogs before and after the initiation of breathing.

FIG. 3 shows charts showing the results of gene expression changes in the diaphragms of normal dogs and of dystrophic dogs before the initiation of breathing (A) and after the initiation of breathing (B) using microarrays.

FIG. 4 shows a chart showing the results of changes in osteopontin expression in the diaphragms of normal dogs and of dystrophic dogs before and after the initiation of breathing by quantitative PCR.

FIG. 5 shows photographs showing the results of changes in osteopontin expression in the diaphragms of normal dogs and of dystrophic dogs before and after the initiation of breathing by Western blotting.

FIG. 6 shows photographs showing the results of changes in osteopontin expression in the diaphragms of normal dogs and of dystrophic dogs before and after the initiation of breathing by immunohistochemistry.

FIG. 7 shows a chart showing the results of changes in gene expression in the diaphragms of dystrophic dogs before and after the initiation of breathing using microarrays.

FIG. 8 shows charts showing the results of changes in expression of c-Fos, EGR1, IL-6, and IL-8 in the diaphragms of normal dogs and of dystrophic dogs before and after the initiation of breathing by quantitative PCR.

FIG. 9 shows photographs showing the results of changes in expression of c-Fos, EGR1, IL-6, and IL-8 in the diaphragms of normal dogs and of dystrophic dogs before and after the initiation of breathing by Western blotting.

FIG. 10 shows photographs showing the results of changes in expression of c-Fos, EGR1, IL-6, and IL-8 in the diaphragms of normal dogs and of dystrophic dogs before and after the initiation of breathing by immunohistochemistry.

### DETAILED DESCRIPTION OF THE INVENTION

The present invention provides novel markers for diagnosis of muscular dystrophy. The expression level of the markers according to the present invention is low in a normal state and high in the case of muscular dystrophy. Accordingly, such markers are useful for diagnosis intended to determine whether or not a patient carries muscular dystrophy, prediction of the development of muscular dystrophy, screening for a therapeutic agent or technique for muscular dystrophy, evaluation of the efficacy of a therapeutic agent or technique for muscular dystrophy, and other purposes.

The present inventors considered that elucidation of pathological mechanisms in newborn dystrophic dogs would lead to elucidation of the mechanisms of myodegeneration. The serum CK levels are high at the time of birth of healthy newborns, although such CK levels are not as high as those in the case of DMD (Rudolph, N., et al., Pediatrics, 1966, 38: 1039-1046; Gilboa, N., et al., Arch Dis Child, 1976, 51: 283-285; Zellweger, H., et al., Pediatrics 1975, 55: 30-34). Since the serum CK levels are lowered by caesarean section, pressure applied to a fetus at the time of delivery from the birth canal, external injuries, or hypoxia is considered to be associated with elevated blood CK levels (Rudolph, 1966; Gilboa, 1976; Drummond L. M. et al., Arch Dis Child 1979; 54: 362-366). Thus, the present inventors examined whether or not stress applied at the time of delivery or the initiation of breathing immediately after birth would be associated with the elevated blood CK levels of newborn dystrophic dogs (see, Example 1).

Subsequently, we examined the genes or molecules with expression levels that would increase in the diaphragms of newborn dystrophic dogs before and after the initiation of breathing. As a result, we found that the expression level of osteopontin increased in dystrophic dogs before the initiation of breathing, compared with the case of normal dogs. While the expression level of osteopontin increased specifically in the diaphragms of newborn dystrophic dogs before the initiation of breathing, it is considered to be activated by intracellular Ca²⁺ increased though the stretch-activated channel, and osteopontin had been activated at a stage prior to myonecrosis, in addition to a regeneration or fibrosis stage. Thus, it is considered to be a molecule associated with the nature of pathological conditions of muscular dystrophy, and it is associated with induction of inflammatory cells (see, Example 2).

In contrast, the expression levels of c-fos and egr-1, which are immediate early genes referred to as “third messengers,” were elevated in dystrophic dogs after the initiation of breathing, compared with the case of dystrophic dogs before the initiation of breathing. Also, the expression levels of the interleukin-6 (IL-6) and interleukin-8 (IL-8) genes located downstream of the above genes were elevated. While these molecules were considered to be activated by significantly increased intracellular Ca²⁺ inflow from mechanically damaged sites of the stretch-activated channel and the muscle cell membrane, IL-6 and IL-8 are also referred to as “myokines,” which are cytokines and chemokines expressed in a muscle cell endogenously and considered to be associated with induction of inflammatory cells, such as neutrophils, occurring at an early stage of muscle damage (see, Example 2).

The present inventors proposed a two-phase hypothesis based on identification of causes for the elevated blood CK levels in newborn dystrophic dogs. That is, the expression level of osteopontin is elevated in the diaphragm before the application of mechanical stress upon initiation of breathing, sarcolemma collapse, and calcium influx in the diaphragm after the application of mechanical stress upon initiation of breathing. Subsequently, the immediate early genes are expressed to increase the expression levels of cytokines and chemokines of IL-6 or IL-8, which are molecules located downstream, and inflammatory cells, such as neutrophils, are induced. The genes and the molecules identified in the research provide a novel perspective on the pathological mechanisms of muscular dystrophy, and such genes and molecules serve as novel molecular markers for evaluation of disease progression or therapeutic effects.

According to the present invention, c-Fos, EGR1, IL-6, and IL-8 proteins with expression levels that are low in a normal state but high in the case of muscular dystrophy, as described above, and genes encoding the proteins are used as markers. Such genes and proteins are referred to as “the markers of the present invention” herein. The markers of the present invention are also referred to as “marker genes” or “marker proteins” herein.

The marker proteins and the marker genes of the present invention are known in the art, and the amino acid sequences and the nucleotide sequences thereof are also known. However, there has been no report regarding any correlation between the markers of the present invention and muscular dystrophy. The names, accession numbers, nucleotide sequences, and amino acid sequences of the markers of the present invention are summarized in Table 1.

TABLE 1

| | | Nucleotide | Amino acid | | Nucleotide | Amino acid |
|---|---|---|---|---|---|---|
| Marker | Accession No. | sequence | sequence | Accession No. | sequence | sequence |
| names | (human) | (human) | (human) | (canine) | (canine) | (canine) |
| c-Fos | K00650 | 1 | 2 | XM_547914 | 3 | 4 |
| EGR1 | NM_001964 | 5 | 6 | XM_846145 | 7 | 8 |
| IL-6 | NM_000600 | 9 | 10 | NM_001003301 | 11 | 12 |
| IL-8 | NM_000584 | 13 | 14 | NM_001003200 | 15 | 16 |

According to the present invention, the markers listed in Table 1 can be used individually. Specifically, c-Fos can be used as a marker in an embodiment. In another embodiment, EGR1 can be used as a marker. In a further embodiment, IL-6 can be used as a marker. In a further embodiment, IL-8 can be used as a marker. Markers listed in Table 1 can be used in combination, according to need. Alternatively, a marker listed in Table 1 may be used in combination with another marker of muscular dystrophy known in the art. Any number of markers can be used in any combination, provided that at least one marker listed in Table 1 is included. Examples of combinations that can be employed include c-Fos and ERG-1; c-Fos and IL-6; c-Fos and IL-8; ERG-1 and IL-6; ERG-1 and IL-8; IL-6 and IL-8; c-Fos, ERG-1, and IL-6; c-Fos, ERG-1, and IL-8; c-Fos, IL-6, and IL-8; and ERG-1, IL-6, and IL-8. An example of another marker includes serum creatinine kinase (CK) level. Use of markers in combination enables more accurate diagnosis of muscular dystrophy.

As described above, the expression levels of such markers are high in the case of muscular dystrophy. According to the present invention, expression of the above marker or the combination of markers in a sample obtained from a subject is determined. When expression of two or more markers is determined, steps of determining the expression of each marker may be carried out simultaneously or sequentially. According to the present invention, “expression of a marker” may be expression of a marker protein, a derivative or precursor thereof, or a gene encoding the protein (mRNA). The term “derivative” or “precursor” refers to a substance derived from a marker protein or a substance from which a marker protein originates. Examples thereof include, but are not limited to, a protein containing a signal peptide, a specific subunit molecule of a protein, a modified protein, and a protein fragment.

Accordingly, the diagnostic agent for muscular dystrophy of the present invention (hereafter, it may be referred to as “the present diagnostic agent”) comprises a means for determining expression of at least one marker selected from the group consisting of c-Fos, EGR1, IL-6, and IL-8 in a sample obtained from a subject.

Any sample can be used, provided that such sample is obtained from a subject to be diagnosed for muscular dystrophy. An adequate sample may be selected depending on a method or means for determining the expression of a marker. Examples include, but not limited to, biological fluid samples (e.g., blood, blood serum, blood plasma, urine, spinal fluid, or ascites) and tissue or cell samples (e.g., muscle tissue or cells, such as diaphragmatic tissue or cells). From the viewpoint of ease of sampling, use of a muscle cell, blood, blood serum, or blood plasma as a sample may be preferable. When plasma sample is used, use of EDTA as an anticoagulant is preferable, and substances known or general in the art, such as heparin or sodium citrate, can be used. In the case of a blood sample, it is preferable that the blood sample be ice-cooled or refrigerated after blood sampling. A tissue or cell sample may be preferably frozen and cryopreserved via a technique known or general in the art, such as with the use of liquid nitrogen or dry ice, immediately after sampling. Muscle cells can be sampled by a method known in the art. Specifically, for example, the skeletal muscle is sliced and transferred to a 50-ml conical bottom tube. Thereafter, 4 ml of a solution of Dispase II (2.4 IU/ml) and Collagenase XI (0.2%) is added per g of muscle, incubated at 37° C. for 45 to 60 minutes, and subjected to pipetting every 15 minutes. Thereafter, tissue slices are pierced with an 18G injection needle several times and grounded, and the supernatant is recovered. The supernatant is applied to a 80-μm filter to remove cell masses, a cell suspension is transferred to a 50-ml conical bottom tube, a growth medium is added to the tube after the treatment with Dispase II (2.4 IU/ml) and Collagenase XI (0.2%), and the supernatant is applied to a filter and recovered in the same manner as described above. A growth medium is added to bring the total amount to 30 ml, and pipetting is carried out several times, followed by centrifugation at 1,000 rpm, 4° C. for 5 minutes. The supernatant is discarded, and 20 ml of growth medium is added to the precipitated cells to resuspend the cells, followed by centrifugation at 1,000 rpm, 4° C. for 5 minutes. The precipitated cells are suspended in 25 ml of growth medium, the suspension is transferred to an uncoated 15-cm culture dish, bFGF is added thereto, culture is conducted at 37° C. in 5% CO₂ for 90 minutes, and the supernatant containing nonadherent cells is recovered. The bottom of the culture dish is washed with the use of the supernatant, the culture dish is turned 180 degrees, culture is conducted again at 37° C. in 5% CO₂ for 90 minutes, and the supernatant is recovered. The recovered supernatant is transferred to a 15-cm collagen-coated culture dish, bFGF is added thereto, and culture is conducted at 37° C. in 5% CO₂ overnight. The culture product is subjected to subculture on the following day if myoblasts reach at least 30% to 40% confluency. If the confluence level is below the aforementioned level, medium exchange is carried out. Thereafter, subculture or medium exchange is carried out every day in order to prevent muscle differentiation.

Subjects may be humans or other mammals, such as primates (e.g., monkeys and chimpanzees), livestock animals (e.g., cattle, horses, pigs, and sheep), pet animals (e.g., dogs and cats), or experimental animals (e.g., mice, rats, and rabbits). Further, subjects may also be reptiles and birds.

Determination of the expression of a marker preferably involves semi-quantitative or quantitative determination of the amounts or concentrations of markers in a sample. Such an amount may be an absolute amount or relative amount. Determination can be carried out directly or indirectly. Direct determination involves determination of the amount or concentration of the marker proteins or genes (mRNAs) existing in a sample based on signals that are directly correlated with the numbers of molecules of such marker proteins or genes. Such signals are based on given physical or chemical characteristics of a protein or gene, for example. Indirect determination involves determination of signals derived from secondary components (i.e., components other than marker proteins or mRNA), such as ligands of antibodies or aptamers, labels, or enzyme reaction products. Determination means used in accordance with the present invention vary depending on methods of determining expression of a marker employed.

In one embodiment of the present invention, marker expression can be determined by a means for determining the marker protein level in a sample. Such means are known in the art, and examples thereof include techniques and reagents for immunoassays. Also, expression of a marker protein can be determined by a means for determining physical or chemical characteristics specific to a marker protein, such as a means for accurately assaying the molecular level or NMR spectra. Examples of means for determining the expression of a marker protein include analyzers, such as biosensors, protein chips, optical devices coupled to immunoassays, mass spectrometers, NMR spectrometers, two-dimensional electrophoresis apparatuses, and chromatography apparatuses.

For example, expression of a marker protein in a sample can be determined by immunoassays (immunological assay techniques). Specifically, expression of a marker protein in the sample can be determined based on the reaction between such protein and an antibody that specifically binds thereto. Immunoassays may be carried out in a liquid phase or solid phase, provided that the technique used is conventional in the art. From the viewpoint of ease of detection, use of a solid phase may be preferable. In addition, immunoassay techniques are not limited, and immunoassay can be carried out by sandwich assay, competitive assay, Western blotting, or enzyme linked immunosorbent assay (ELISA), as well as a direct solid-phase assay.

When immunoassay techniques are adopted, the present diagnostic agent comprises an antibody against a marker protein. An antibody against a marker protein may be a monoclonal or polyclonal antibody. Alternatively, it may be, for example, an Fab or Fv fragment capable of binding to an epitope of a marker protein. When a primary antibody and a secondary antibody are used, both thereof may be monoclonal antibodies. Alternatively, either the primary or secondary antibody may be a polyclonal antibody. An antibody can be prepared by a method known in the art or a commercially available antibody may be used.

Binding (reaction) between a marker protein and an antibody can be assayed in accordance with a method well-known in the art. A person skilled in the art can determine an effective and optimal assay technique in accordance with the type, format, type of labels to be used, and other conditions of the immunoassay to be adopted. In order to easily detect binding between a marker protein in a sample and an antibody, for example, the binding can be directly detected by labeling the antibody or indirectly detected with the use of, for example, a labeled secondary antibody or a biotin-avidin complex.

When a solid-phase immunoassay is selected, for example, a protein component in a sample can be immobilized to a solid phase. An example of a method that can be adopted is a method comprising: (1) preparing a protein component from a sample; (2) fractionation via SDS-polyacrylamide gel electrophoresis; (3) transferring a protein on a gel to a solid phase; (4) reacting the solid phase with an antibody against the marker protein (a primary antibody); (5) washing the solid phase; (6) contacting a labeled antibody against the primary antibody (a secondary antibody) with the solid phase; (7) washing the solid phase; and (8) assaying the expression level of the protein based on the label. Alternatively, an antibody may be immobilized onto a solid phase. This method is referred to as a so-called “sandwich assay” method, which is extensively employed for “ELISA” when an enzyme is used as a marker. Such solid-phase technique may be preferable for detection of trace amounts of proteins and simplification of procedures.

In a solid-phase system, an antibody or a protein component in a sample may be immobilized onto a solid phase (e.g., a plate, membrane, or bead), and immunological binding between a marker protein and an antibody may be tested on the solid phase. Any solid phase that is conventionally used in the art can be used without particular limitation. For example, a commercially available nitrocellulose membrane or PVDF membrane can be used. By immobilizing an antibody or a protein component in a sample onto a solid phase, an unbound sample component or reagent can be easily removed. In the case of protein array techniques involving the use of a membrane onto which several types of antibodies have been immobilized, in particular, expression of a plurality of marker proteins can be analyzed within a short period of time with the use of a small amount of a sample obtained from a subject (e.g., a blood plasma sample). Such immunoassays can be carried out via, for example, test strip assays that are easy to operate.

When a liquid-phase immunoassay system is selected, for example, a labeled antibody may be contacted with a sample to bind the labeled antibody to a marker protein, the resulting complex is separated, and a labeled signal is detected. Alternatively, an antibody against a marker protein (a primary antibody) may be contacted with a sample to bind the primary antibody to a marker protein, a labeled antibody (a secondary antibody) is allowed to bind to the resulting complex, and a labeled signal in the complex of such three components is then detected. In order to further potentiate signals, a nonlabeled secondary antibody is first allowed to bind to a complex of an antibody and a marker protein, and a label may then be bound to the secondary antibody. A label can be bound to a secondary antibody by, for example, biotinylating a secondary antibody and avidinylating a label.

Antibodies used in immunoassays can be labeled with enzyme, radioisotope, fluorescent dye, or avidin-biotin system. Enzymes used for conventional enzyme immunoassays (ETA), such as peroxidase, β-galactosidase, or alkaline phosphatase, can be used. Enzyme inhibitors, coenzymes, or the like can also be used. Such enzymes can be bound to antibodies in accordance with a conventional technique involving the use of a crosslinking agent, such as a maleimide compound. Radioisotopes, such as ¹²⁵I or ³H, that are used for conventional radioimmunoassay (RIA) can be used. Fluorescent dyes, such as fluorescein isothiocyanate (FITC) or tetramethylrhodamine isothiocyanate (TRITC), that are used for conventional fluorescent antibody techniques can be used.

When a biotin-avidin conjugate is used, a biotinylated antibody is allowed to react with a sample, and labeled avidin is then allowed to react with the resulting conjugate. Since avidin is capable of specifically binding to biotin, binding between an antibody and a marker protein can be determined by detecting a signal emitted from the label added to avidin. A label added to avidin is not particularly limited, and an enzyme label, such as peroxidase or alkaline phosphatase, may be preferable.

A signal of a label can also be detected in accordance with a method known in the art. When an enzyme label is used, for example, a substrate that develops color upon degradation caused by enzymatic action is added, the amount of the substrate degraded is optically assayed to determine the enzyme activity, the determined value is converted to yield the amount of bound antibody, and the obtained value is compared with the reference value. Thus, the amount of antibody is determined. Different substrates can be used in accordance with the type of enzyme to be used. When peroxidase is used as an enzyme, for example, 3,3′,5,5′-tetramethylbenzidine can be used. When alkaline phosphatase is used as an enzyme, for example, paranitrophenol can be used. When a radioactive label is used, a radiation dose emitted by a radioactive label may be assayed with the use of a scintillation counter or the like. A fluorescent label can be detected and quantified with the use of, for example, a fluorescence microscope or plate reader.

In order to detect a marker protein in situ as in the case of immunohistochemical staining (e.g., immunostaining) or immune electron microscopy, an antibody against a marker protein can be used in these methods. In situ detection can be carried out by resecting histological samples (e.g., muscle tissue, muscle cell, or diaphragmatic samples) from a subject (e.g., slices of paraffin-embedded tissue) and bringing a labeled antibody into contact therewith.

According to the immunological techniques described above, the expression levels of marker proteins increase in a sample as the amounts of antibodies bound to marker proteins increase in a sample.

Alternatively, the expression of a marker protein can be determined by mass spectrometry (MS). Analysis by liquid chromatography coupled with mass spectrometry (LC/MS) is accurate and thus is particularly advantageous. Mass spectrometry analysis can be carried out by, for example, (1) preparing a protein component from a sample, (2) labeling proteins or peptides; (3) fractionating proteins or peptides, (4) subjecting proteins or peptides to mass analysis, and (5) identifying marker proteins based on the values obtained by mass spectra. Isotopic labeling reagents known in the art can be used as labels, and adequate labeling reagents are commercially available. Also, fractionation can be carried out by a method known in the art. For example, it can be carried out with the use of commercially available strong cation-exchange columns. In such a case, the present diagnostic agent comprises isotopic labeling reagents, mini columns for fractionation, and the like as means for determining the expression of a marker.

According to the present invention, the determination of marker expression can be carried out by a means for determining the amount of marker genes in a sample. Such means is known in the art. Examples thereof include primer DNA or probe DNA containing or consisting of all or part of the DNA sequence of a marker gene or a sequence complementary thereto. Such primer DNA or probe DNA specifically binds to mRNA of the marker gene expressed in a sample obtained from a subject or cDNA corresponding to such mRNA, and it is capable of detecting marker gene expression in a sample.

Primer DNA or probe DNA can be readily designed based on the nucleotide sequence of DNA of a marker gene with the use of a known program, and primer DNA or probe DNA can be prepared in accordance with a method known in the art. Specifically, primer DNA or probe DNA can be designed based on the nucleotide sequence of the marker gene, such as the nucleotide sequence as shown in SEQ ID NO: 1, 3, 5, 7, 9, 11, 13, or 15 or a sequence complementary thereto. DNA that substantially functions as a primer preferably comprises 10 or more nucleotides, more preferably 15 to 50 nucleotides, and further preferably 20 to 30 nucleotides. Also, DNA that substantially functions as a probe preferably comprises 10 or more nucleotides, more preferably 15 to 50 nucleotides, and further preferably 20 to 30 nucleotides. Primer DNA or probe DNA may comprise an additional sequence other than a region that may anneal or hybridize to a marker gene, such as a tag sequence, as is well-known in the art.

In order to determine the expression of a marker gene in a sample obtained from a subject, the above-described primer DNA and/or probe DNA may be used in amplification or hybridization, and the amplification or hybridization product may be detected. In such a case, in general, mRNA or cDNA corresponding thereto may be prepared from a sample obtained from a subject by a method well-known in the art. When RNA is to be extracted, for example, guanidine-cesium chloride ultracentrifugation, the hot-phenol method or the acid guanidium thiocyanate-phenol-chloroform (AGPC) method can be employed. cDNA can be prepared with the use of a known reverse transcriptase. The thus-obtained sample may be used to carry out the amplification and/or hybridization reactions described below.

The expression of a marker gene in a sample can be determined by carrying out an amplification using primer DNA and mRNA or cDNA as a template, and detecting specific amplification. Amplification techniques are not particularly limited. For example, known techniques based on the principle of polymerase chain reactions (PCR), such as PCR, RT-PCR, or real-time PCR, can be employed. An amplification product can be detected by a known means that is capable of specifically recognizing an amplification product. For example, whether or not a fragment of a certain size is amplified may be determined by agarose gel electrophoresis or other means to detect a specific amplification reaction.

Alternatively, a label, such as a radioisotope, fluorescent substance, or luminescent substance, may be added to dNTP that is to be incorporated during amplification, and the resulting labeled substance can be detected. Examples of radioisotopes that can be used include ³²P, ¹²⁵I, and ³⁵S. Fluorescent substances, such as fluorescein isothiocyanate (FITC), sulforhodamine (SR), and tetramethylrhodamine isothiocyanate (TRITC), can be used. Luciferin or the like can be used as a luminescent substance. Types of label, methods of introducing a label, and others are not particularly limited, and various conventional means can be employed. An example of a method of introducing a label is a random priming method involving the use of a radioisotope.

An amplification product into which labeled dNTP has been incorporated may be observed by any technique for detecting such a label that is known in the art. When a radioisotope is used as a label, for example, radioactivity can be measured by a liquid scintillation counter or γ-counter. When a fluorescent substance is used as a label, for example, fluorescence can be detected by a fluorescence microscope or fluorescence plate reader.

The expression of a marker gene can be determined by subjecting probe DNA to hybridization to a sample, and detecting specific binding (hybridization). It may be necessary to carry out hybridization under conditions where probe DNA specifically and selectively binds to mRNA or cDNA of the marker gene in a sample (i.e., stringent conditions). When hybridization is carried out, an adequate label, such as a fluorescent label (e.g., fluorescein or rhodamine), radioactive label (e.g., ³²P), or biotin label, can be added to probe DNA.

Detection involving the use of labeled probe DNA comprises contacting a sample or either mRNA or cDNA prepared therefrom with probe DNA, so as to allow hybridization to take place. Specifically, a sample or either mRNA or cDNA may be immobilized onto an adequate solid phase, and labeled probe DNA may be applied thereto. Alternatively, labeled probe DNA may be immobilized onto an adequate solid phase, and a sample or either mRNA or cDNA may be applied thereto. Thus, probe DNA is brought into contact with a sample or either mRNA or cDNA to carry out hybridization, unhybridized probe DNA is removed, and a label of the probe DNA that has hybridized to a sample or either mRNA or cDNA is then detected. The detection of a label indicates that mRNA of a marker gene is expressed in a sample. Examples of expression assay techniques involving the use of labeled probe DNA include Southern hybridization and Northern hybridization.

As described above, marker expression in a sample can be determined with the use of a means for determining expression of a marker, and muscular dystrophy can be diagnosed based on the results. The disease to be diagnosed with the use of the present diagnostic agent is muscular dystrophy, and, in particular, Duchenne muscular dystrophy. The term “diagnosis of muscular dystrophy” used herein indicates determination of carrier of muscular dystrophy or prediction of development of muscular dystrophy in a subject. According to the present invention, the term “diagnosis” may also encompass continuous monitoring of muscular dystrophy that has already been diagnosed and confirmation of previous diagnosis of muscular dystrophy.

Diagnosis in accordance with the present invention is not intended to always yield accurate results for all subjects to be diagnosed (i.e., 100%). The present invention is intended to diagnose subjects with statistically significant accuracy. For example, 60% or more, preferably 80% or more, or more preferably 90% or more of the subjects can be adequately diagnosed according to the present invention.

At the time of diagnosis, marker expression in a sample obtained from a subject is compared with a reference value. A reference value may be, for example, a marker expression level determined in a sample obtained from a healthy individual or a marker expression level determined in a sample obtained from a patient who has been diagnosed as being a carrier of or having developed muscular dystrophy. The reference value adopted for each subject varies depending on various biological parameters, such as a marker type, a subject type, age, and other factors. When the marker expression level in a sample obtained from a subject is greater in 10% or more, preferably 30% or more, more preferably 70% or more, and most preferably 100%, compared with that in a sample obtained from a healthy individual, specifically, a subject can be diagnosed as being likely to have muscular dystrophy.

Further, diagnosis of muscular dystrophy may be carried out in combination with other known techniques for diagnosing muscular dystrophy. Examples of known diagnostic techniques include measurement of serum creatine kinase (CK) levels, tension measurement of isolated skeletal muscle, histological measurement of the maximal diameter of muscles and frequency of centronuclear fibers, multiplex ligation-dependent probe amplification (MLPA), identification of gene mutation by polymerase chain reaction (PCR)/sequencing, immunohistochemical techniques involving the use of anti-dystrophin antibodies, and qualitative and quantitative analysis of the dystrophin protein by Western blotting.

The present diagnostic agent may comprise other components useful for diagnosis, in addition to a means for determining expression of a marker. This enables easy and simple assay of marker expression and diagnosis of muscular dystrophy.

An example of the present diagnostic agent is a set of reagents for immunoassays that at least comprises an antibody reagent for a marker protein. In addition, the set may comprise a buffer for dilution or washing, a standard antigen, a labeled antibody reagent that specifically binds to an antibody reagent, substrate reagents that develop color, luminescence or fluorescence, and instructions describing procedures and evaluation methods. An antibody included in the set may be labeled in advance, or it may not be labeled. In addition, an antibody may be immobilized onto a solid-phase support (e.g., a membrane or bead). Another example of the diagnostic agent according to the present invention is a set of reagents for mass analysis, which is composed of, for example, isotopic labeling reagents, mini columns for fractionation, buffer, and instructions. A further example of the diagnostic agent according to the present invention may comprise a means for determining expression of a marker gene in a sample (e.g., primer DNA or probe DNA).

The diagnostic agent according to the present invention may comprise instructions describing procedures and protocols for use, a table showing reference values or ranges used for diagnosis of muscular dystrophy, and other components.

Components included in the diagnostic agent according to the present invention may be provided separately or in a single container. Preferably, the diagnostic agent according to the present invention comprises all components adjusted at concentrations that allow all the necessary components to be used immediately.

In addition, the efficacy of a therapeutic agent or technique for muscular dystrophy can be evaluated, and a candidate for the therapeutic agent or technique for muscular dystrophy can be screened with the use of the marker(s) described above. Specifically, an animal that had developed muscular dystrophy, a muscular dystrophy carrier animal, or a muscle cell derived from an animal that had developed muscular dystrophy or a muscular dystrophy carrier animal (e.g., the diaphragmatic cell) may be treated with a test agent or technique, and marker expression in such animals or muscle cells may be determined. Thus, whether or not the test agent or technique affects marker expression (i.e., muscular dystrophy) can be determined.

According to the screening method of the present invention (hereafter, it may be referred to as “the present screening method”) and the method for evaluating the efficacy of a therapeutic agent or technique (hereafter, it may be referred to as “the present evaluation method”), an animal that had developed muscular dystrophy or a muscular dystrophy carrier animal is first treated with a test agent or technique. Alternatively, a muscle cell derived from an animal that had developed muscular dystrophy or a muscular dystrophy carrier animal is first treated with a test agent or technique.

In the present method, a sample may be obtained from an animal that had developed muscular dystrophy or a muscular dystrophy carrier animal, and marker expression in the sample may be determined. Alternatively, marker expression in the muscle cell may be determined. Preferably, a sample may be obtained from an animal that had developed muscular dystrophy or a muscular dystrophy carrier animal prior to the treatment with a test agent or technique, and marker expression in the sample or in the muscle cell may be determined. An animal that had developed muscular dystrophy, a muscular dystrophy carrier animal, or a muscle cell derived from an animal that had developed muscular dystrophy or a muscular dystrophy carrier animal may be treated with the test agent or technique, and marker expression in the sample or cell may be determined at appropriate times. For example, marker expression may be determined immediately after treatment or 30 minutes, 1 hour, 3 hours, 5 hours, 10 hours, 15 hours, 20 hours, 24 hours (1 day), 2 to 10 days, 10 to 20 days, 20 to 30 days, and/or 1 month to 6 months after treatment. Sample collection and determination of marker expression in a sample can be carried out in the same manner as described above.

Animals to be tested may be humans who have developed muscular dystrophy or are muscular dystrophy carriers, or animal models of muscular dystrophy, and experimental animal models of muscular dystrophy (e.g., mice, dogs, and rats) are preferable. Examples of experimental animals that can be employed include mouse models of muscular dystrophy (mdx mice: Sicinski, P. et al., Science 244: 1578-1580, 1989), dog models of muscular dystrophy (c-xmd dogs: Komegay, J. N. et al., Muscle Nerve 11, 1056-1064, 1988; CXMD_{J} dogs: Shimatsu, Y. et al., Exp. Anim. 52, 93-7. 2003, Shimatsu, Y. et al., Acta Myol. 24, 145-54, 2005), and cat models of muscular dystrophy (HFMD cats: Vas, J. H. et al., J. Comp. Pathol., 96, 335-41, 1986). In general, the efficacy of a test agent or technique may be first verified in animal models, and it is then evaluated via, for example, clinical trials in humans.

Test agents or techniques subjected to the screening method and the evaluation method of the present invention are not particularly limited. For example, the test agent or technique include: any physical factors, and specifically, naturally occurring molecules, such as amino acids, peptides, oligopeptides, polypeptides, proteins, nucleic acids, lipids, carbohydrates (e.g., sugar), steroids, glycopeptides, glycoproteins, and proteoglycans; synthetic analogues or derivatives of naturally occurring molecules, such as peptide mimics and nucleic acid molecules (e.g., aptamers, anti-sense nucleic acids, and double-stranded RNA (RNAi)); non-naturally occurring molecules, such as low-molecular-weight organic compounds prepared with the use of combinatorial chemistry techniques (e.g., a library of inorganic and organic compounds or a combinatorial library); and a mixture of any thereof. Moreover, the test agent or technique may involve the use of a single substance, a complex constituted by a plurality of substances, a transcription factor, or the like. Furthermore, the test agent or technique may involve the use of, for example, radiation or ultraviolet light, in addition to the physical factors described above.

Moreover, a single test agent or technique may be independently examined, or a combination of several candidate agents or techniques (including in the form of libraries or the like) may be examined. Examples of libraries containing a plurality of test agents or techniques to be tested include a library of synthetic compounds (e.g., a combinatorial library) and a peptide library (e.g., a combinatorial library).

When an animal is treated with a test agent or technique, conditions for treatment such as the dose for treatment, treatment period, and route of treatment vary depending on the type of test agent or technique. A person skilled in the art can easily determine such conditions. When the test agent is administered to an animal, for example, administration routes such as intramuscular injection, oral administration, intravenous injection, intraperitoneal injection, transdermal injection, or subcutaneous injection may be appropriately selected in accordance with the type of test agent, the type of animal to be used, and other conditions.

Muscle cells may be collected according to methods known in the art, such as the method described above. Alternatively, muscle cells that are commercially available or available to the public can be used. When cells are exposed to the test agent or technique, the conditions for exposure vary depending on the type of test agent or technique; however, a person skilled in the art can easily determine such conditions. For example, such exposure may be performed by culturing the muscle cells in a medium supplemented with the test agent, immersing the muscle cells in a solution containing the test agent, or overlaying the test agent on the muscle cells.

Furthermore, the effects and the efficacy of the test agent or technique may be examined under various conditions. Examples of such conditions include the time or period, the amount (large or small), and the frequency of the treatment with the test agent or technique. For example, a plurality of doses may be set by preparing a dilution series of test agents. The period of treatment with the test agent or technique can also be appropriately set, and the treatment may be preformed for a period of 1 day to several weeks, several months, or several years, for example.

Furthermore, when the additive action, synergistic action, or the like of a plurality of test agents and/or test techniques are examined, such plurality of test agents and/or test techniques may be used in combination.

Subsequently, marker expression in an animal model or cell may be determined. Marker expression can be determined in the manner as described above. After marker expression is determined, the determined value may be compared with a control sample, and a test agent or technique that lowers marker expression levels may be selected. Animals or cells that are not treated with the test agent or technique can be used as controls.

According to the present invention, muscle cells may be treated with the test agent or technique for the primary screening, the test agent or technique that exhibits the lower marker expression level in the muscle cells may be selected. Subsequently, secondary screening may be carried out by treating animals with the selected test agent or technique and determining marker expression in such animals to select the test agent or technique that exhibits the lower expression level.

When screening for a therapeutic agent or technique, further, a selected test agent may be administered to animal models of muscular dystrophy, or the animals may be subjected to the a selected test technique to determine whether or not the test agent or technique would affect the development, progression, or symptoms of muscular dystrophy in animal models. The results of such determination vary depending on the type of animal model, symptoms to be diagnosed, various factors, or other conditions. However, a person skilled in the art would be able to adequately determine influences imposed on muscular dystrophy. When influences imposed on muscle diseases or myopathy are to be assayed, for example, measurement of muscle strength, measurement of serum creatine kinase levels, tension measurement of isolated skeletal muscles, and histological measurement of the maximal diameter of muscles and frequency of centronuclear fibers can be carried out.

When amelioration of muscular dystrophy is observed (e.g., amelioration of symptoms or delay of disease development or progression) as described above, the test agent or technique can be selected as a candidate for therapeutics for treatment or prevention of muscular dystrophy.

Thus, the present screening method and the present evaluation method enable identification of the therapeutic agent or technique used for treatment or prevention of muscular dystrophy and verification of efficacy of such therapeutic agent or technique.

Since the expression level of c-Fos, EGR1, IL-6, or IL-8 is elevated at the time of disease development (after birth), expression or activity of at least one member selected from the group consisting of c-Fos, EGR1, IL-6, and IL-8 may be inhibited to treat or prevent muscular dystrophy. Accordingly, the present invention also relates to an agent for treating or preventing muscular dystrophy comprising a means for inhibiting or suppressing expression or activity of at least one member selected from the group consisting of c-Fos, EGR1, IL-6, and IL-8. Examples of such means include means for inhibiting or suppressing gene expression, such as methods involving the use of anti-sense nucleic acids or RNAi (e.g., micro RNA or siRNA), and means for inhibiting or suppressing protein expression, such as antibodies and nucleic acid aptamers.

### EXAMPLES

Hereafter, the present invention is described in greater detail with reference to the following examples and the drawings, although the present invention is not limited to the examples below.

### Example 1

### Changes in Serum Creatinine Kinase (CK) Level

It has been reported that stress during labor may result in the development of myopathy in newborns, and that such stress may be reduced by caesarean section. Based on these reports, elective caesarean section was adopted to examine differences in serum CK levels after spontaneous labor and after caesarean section in normal dogs, carrier dogs, and dystrophic dogs. The term “carrier dogs” refers to female dogs having a mutation in the dystrophin gene. Specifically, human and dog chromosomes consist of 23 pairs of 46 chromosomes in total. Among these pairs, 22 pairs are autosomal chromosomes, and there is 1 pair of two sex chromosomes that determine the gender. While male dogs (males) have X- and Y-chromosomes, female dogs (females) have two X-chromosomes. The dystrophin gene, which is a causal gene for muscular dystrophy, is present on the X-chromosome and it exhibits X-linked inheritance. Thus, a male individual having a mutation in the X-chromosome develops muscular dystrophy. A female individual having a mutation in the dystrophin gene on one of the two X-chromosomes is referred to as a “carrier dog (carrier).” A dystrophic dog results from crossing such a carrier dog with a normal male dog. In general, carrier dogs exhibit no symptoms, but carrier dogs sometimes exhibit intermediate symptoms between dystrophic dogs and normal dogs. Such a carrier dog is referred to as a “symptomatic carrier dog.”

As test dogs, there were 71 normal dogs, 37 carrier dogs, and 34 dystrophic dogs obtained via spontaneous labor (39 times) and 39 normal dogs, 26 carrier dogs, and 41 dystrophic dogs obtained via elective caesarean section (28 times) from December, 2001, to April, 2008, at the dystrophic dog breeding colony of the Mid-sized Animal Research Facility, the National Institute of Neuroscience, the National Center of Neurology and Psychiatry (NCNP). Elective caesarean section was carried out on a date predicted based on the LH Surge (Witness® LH, Synbiotics, Kansas City, Mo., U.S.A.) or when the body temperature of a pregnant carrier dog rapidly dropped (Kobayashi, M., et al., Muscle Nerve, 2009, 40: 815-826). Caesarean section of a pregnant carrier dog was carried out with the use of isoflurane (2.0% to 3.0%) from induction to maintenance of anesthesia. Newborn dogs were subjected to resuscitation by a veterinarian or a licensed animal handling technician who has experience in resuscitation under the supervision of a veterinarian. A respiratory stimulant (i.e., doxapram) was used for each dog after resuscitation, and the dog was laid on a dry, warm towel in a box supplemented with oxygen.

In order to detect changes in serum creatinine kinase (CK) levels over time (the umbilical cord blood and 30 minutes, 1 hour, 2 hours, 4 hours, 8 hours, 24 hours, and 48 hours after resuscitation), 5 normal dogs, 3 carrier dogs, and 6 dystrophic dogs obtained via elective caesarean section (3 times) were used. Four dogs from each group obtained via caesarean section were subjected to pathological and molecular biological analyses. This research was approved by the Committee on Mid-sized Animal Ethics, the National Institute of Neuroscience, the National Center of Neurology and Psychiatry (Approval numbers: 13-03, 14-03, 15-03, 16-03, 17-03, 18-03, 19-04, and 20-04) and conducted in accordance with the animal experiment guidelines thereof.

The serum creatinine kinase (CK) levels were determined by subjecting the umbilical cord blood or venous blood to centrifugation at room temperature, 1,800 g for 10 minutes to separate the blood serum, followed by colorimetry (FDC3500, FujiFilm, Tokyo, Japan).

After euthanasia, the diaphragm was freeze-fixed, cut into 7 μm slices, and then subjected to hematoxylin-eosin (H & E) staining and calcium staining (i.e., alizarin red staining) (pH 4.1).

Data for 2 groups were compared by the student-t test. The chi-square test was carried out to determine the mortality rate. Data were expressed as average±standard deviation and considered to be significantly different by p<0.05.

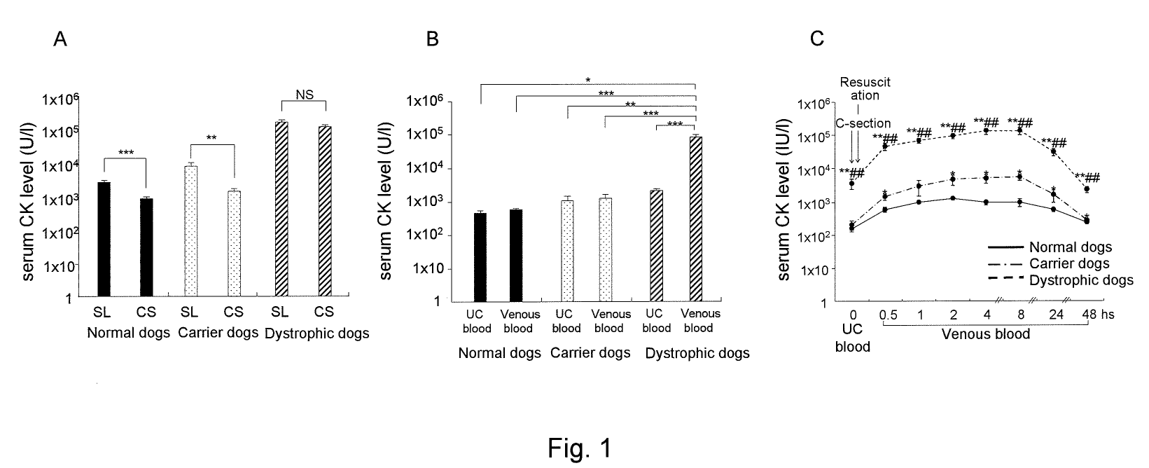

As a result, the serum CK levels were found to have decreased significantly in normal dogs and carrier dogs after caesarean section, although no decrease was observed in dystrophic dogs (FIG. 1A). The above results suggest that stress during labor is not a primary cause for the elevated blood CK levels of newborn dystrophic dogs. Since newborns undergo the important breathing initiation process immediately after birth, whether or not the cause of the elevated blood CK levels is associated with the initiation of pulmonary breathing was examined by comparing the serum CK levels in the umbilical cord blood of newborns obtained via caesarean section (reflecting conditions before the initiation of breathing) and in the jugular venous blood 1 hour after the initiation of breathing. No differences between the serum CK levels and the CK levels in the umbilical cord blood of normal dogs and carrier dogs after the initiation of breathing were observed.

While the CK levels in the umbilical cord blood of dystrophic dogs were approximately 5 times higher than those of normal dogs, the CK levels in venous blood of dystrophic dogs after the initiation of breathing were approximately 35 times higher than those in the umbilical cord blood of dystrophic dogs and approximately 150 times higher than those in the venous blood of normal dogs after the initiation of breathing (FIG. 1B). In all dog groups, the serum CK levels elevated rapidly up to 30 minutes after the initiation of breathing, reached to a peak 4 to 8 hours after the initiation of breathing, and returned to levels equivalent to those in the umbilical cord blood 48 hours later. The serum CK levels remained high in dystrophic dogs (FIG. 1C).

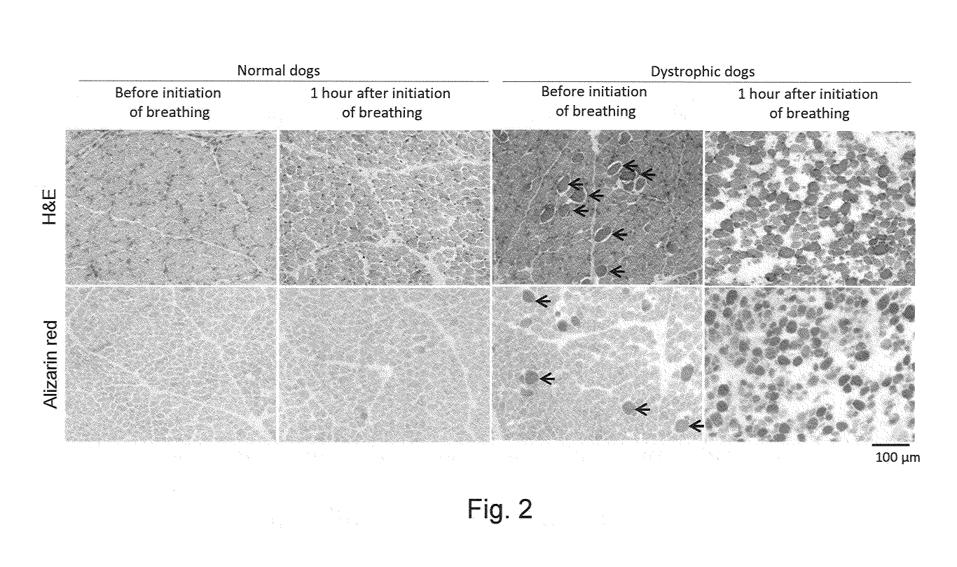

According to a pathological test of the diaphragm before the initiation of breathing, calcium-positive, opaque fibers were occasionally observed (indicated by arrows), and the fundamental muscular structure was maintained. However, after the initiation of breathing, many opaque calcium-positive fibers were observed (indicated by arrows), the interstitium was increased, hyaline degeneration was observed, and substantially no invasion of inflammatory cells (e.g., neutrophils) was observed (FIG. 2).

Based on the above results, rapid mechanical stress imposed by the initiation of breathing was considered to have caused significant myopathy, including the tearing of muscular fibers in the diaphragm. In general, respiratory disorders are not observed at birth in case of DMD; however, it is reported that respiratory disorders frequently occur in premature newborns (Phadek, A, et al., Anesth Analg., 2007, 105: 977-980). Since the muscular development of dogs, including dystrophic dogs, takes place at a significantly slower rate than that of other animals or humans (Lanfossi, M., et al., Acta Neuropathol., 1999, 97: 127-138), the different respiratory disorder development timing between dystrophic dog and DMD is considered to be associated with differences in muscular maturation at birth. Based on the observation of serum CK levels over time, the serum CK levels for all dogs returned to the levels in the umbilical cord blood 48 hours after birth, although dystrophic dogs maintained high serum CK levels. This suggests that influence of labor observed in normal dogs and carrier dogs is eliminated at least 2 days after birth and that screening of newborns with the use of serum CK levels at this point may be feasible. Early diagnosis of DMD is considered to be important for future family plans or for the examination of therapeutic methods that may be developed in the future at an early stage. Accordingly, determination of the serum CK levels at various time point during the neonatal period may achieve a novel finding regarding the timing for screening of newborns with DMD.

### Example 2

### Changes in Gene Expression in the Diaphragm

In this Example, genes with expression levels that increased in the diaphragms of dystrophic dogs after the initiation of breathing compared to that before the initiation of breathing were examined with the use of cDNA microarrays. Specifically, neonatal normal dogs and dystrophic dogs (4 individuals each) were delivered via caesarean section before the initiation of breathing and subjected to autopsy before resuscitation. In addition, neonatal normal dogs and dystrophic dogs (4 individuals each) that had already initiated breathing were subjected to autopsy 1 hour after resuscitation. Total RNA was extracted from the sampled and cryopreserved diaphragm with the use of the RNeasy Mini Kit (Qiagen, Hilden, Germany). RNA concentration was measured using NanoDrop ND-1000 UV-spectrophotometer (NanoDrop Technologies, Wilmington, Del., U.S.A.). RNA quality was examined by Agilent Bioanalyzer 2100 (Agilent Technologies, Santa Cruz, Calif., U.S.A.). Total RNA (500 ng) was applied to the dog whole genome oligo microarray 44K (Agilent Technologies), and hybridization was carried out at Bio Matrix Research, Inc. (Nagareyama, Chiba). A fluorescent image was obtained with the use of the Agilent Technologies microarray scanner (Agilent Technologies). Normalization was carried out by comparing genes on different chips with the use of GeneSpring 10.0 (Torry Digital Biology, Denver, Colo., U.S.A.). Genes exhibiting different expression levels before the initiation of breathing and after the initiation of breathing of normal dogs and dystrophic dogs were subjected to ANOVA test and then selected via the multiple-group test by the Benjamini and Hochberg method.

Based on the results of microarray analysis, genes exhibiting expression levels that were elevated by 10 times or greater compared with other groups were tested via quantitative PCR (real-time PCR). The total RNA of each dog was the same as that used for cDNA microarray analysis. Amplification primers for the 18s RNA (the internal control), osteopontin, c-fos, egr-1, IL6, and IL8 genes were designed (Table 2).

TABLE 2
Primer sequences for real-time RT-PCR

| Genes | forward (5′ > 3′) | reverse (5′ > 3′) |
|---|---|---|
| 18sRNA | GGAAAGTACAGCCAGGTCC | ACACGAAGTCCCCAAAAGTG |
| | (SEQ ID NO: 17) | (SEQ ID NO: 18) |
| Osteopontin | ACGATGTGGATAGCCAGGAC | GGACGGCATTGAAGTCATCT |
| | (SEQ ID NO: 19) | (SEQ ID NO: 20) |
| c-fos | ACTCCAGGGCTGGCGTTGTG | AGTCAGCTCCCTCCTGCGGT |
| | (SEQ ID NO: 21) | (SEQ ID NO: 22) |
| EGR1 | GACAACCACCTTTTCTCCCA | GGCAGTAGGAACTGCAGAGG |
| | (SEQ ID NO: 23) | (SEQ ID NO: 24) |
| IL-6 | GCTACTGCTTTCCCTACCCC | TTTTCTGCCAGTGCCTCTTT |
| | (SEQ ID NO: 25) | (SEQ ID NO: 26) |
| IL-8 | AGAGTGATTGACAGTGGCCC | ACACCAGGTCTACACGGGAC |
| | (SEQ ID NO: 27) | (SEQ ID NO: 28) |

Real-time PCR was carried out using the SYBR mixed Ex Taq II kit (Takara) by repeating a PCR cycle of 95° C. for 20 seconds and 60° C. for 1 minute 40 times in the BioRad iCycler system (BioRad). Gene expression levels were determined relative to 18s RNA ((Ct/18s RNA—Ct/target genes)), and the expression levels of groups of normal dogs and dystrophic dogs were compared before and after the initiation of breathing.

In addition, proteins corresponding to the genes were subjected to Western blot analysis and protein assay via an immunohistochemistry. Western blot analysis was carried out as described below. The freeze-fixed diaphragm was homogenized in a sample buffer (62.5 mM Tris-HCl, pH 6.8, 2% SDS, 10% glycerol), and centrifuged at 15,000 g for 10 minutes, and the supernatant was then recovered. After protein concentration was assayed using the DC Assay Kit (BioRad, CA, U.S.A.), proteins were subjected to thermal denaturation at 95° C. for 5 minutes, and 40 μg of the resultant was subjected to Western blot analysis. A PVDF transfer membrane was blocked with 0.1% Tween 20-containing Iris-buffered saline (TBST)+5% skimmed milk, and incubated with primary antibodies at 4° C. for 16 hours. As primary antibodies, Osteopontin (Rb-9097, Thermo Fisher Scientific), c-Fos (#2250, Cell Signaling Technology), EGR1 (sc-189, Santa Cruz Biotechnology), IL-6 (AF1609, R&D Systems, Minneapolis, Minn., U.S.A.), and IL-8 (ab34100, Abeam) were used. After the transfer membrane was washed with a TBST solution, the membrane was incubated with secondary antibodies (mouse- or rabbit-specific HRP-labeled antibodies) and washed again with a TBST solution, and detection was carried out with the use of the ECL-Plus Western Blotting Detection System (GE HealthCare, Buckinghamshire, UK).

Immunohistochemistry was carried out by drying the sliced freeze-fixed sample for 15 minutes, washing the resultant with phosphate buffer saline (PBS) (pH 7.4) containing 5% bovine serum albumin (BSA) or heat-inactivated normal goat serum albumin, and incubating the samples with primary antibodies at 4° C. for 16 hours. Primary antibodies used were CD18 (MCA1780, AbD Serotec, Oxford, UK), CD68 (M0876, Dako, Denmark), CD11b (MCA1777S, AbD Serotec), C5b-9 (ab66768, Abeam, Cambridge, UK), cleaved-caspase 3 (#9661, Cell Signaling Technology, Beverly, Mass., U.S.A.), LC3 (#4108, Cell Signaling Technology), osteopontin (Rb-9097, Thermo Fisher Scientific, Waltham, Mass., U.S.A.), c-Fos (#2250, Cell Signaling Technology), EGR1 (#4153, Cell Signaling Technology), IL-6 (sc-80108, Santa Cruz Biotechnology, Santa Cruz, Calif., U.S.A.), and IL-8 (109-401-311, Rockland Immunohistochemical, Gilbertsville, Pa., U.S.A.). The samples were washed with PBS at room temperature, incubated with FITC-labeled secondary antibodies at room temperature, washed again with PBS, and then observed under a fluorescence microscope.

Data for 2 groups were compared by the student-t test. The chi-square test was carried out to determine the mortality rate. For comparison of data obtained by real-time PCR among multiple groups, the ANOVA test and then the multiple-group test based on the Tukey's method were performed. Data were expressed as average±standard deviation and considered to be significantly different by p<0.05.

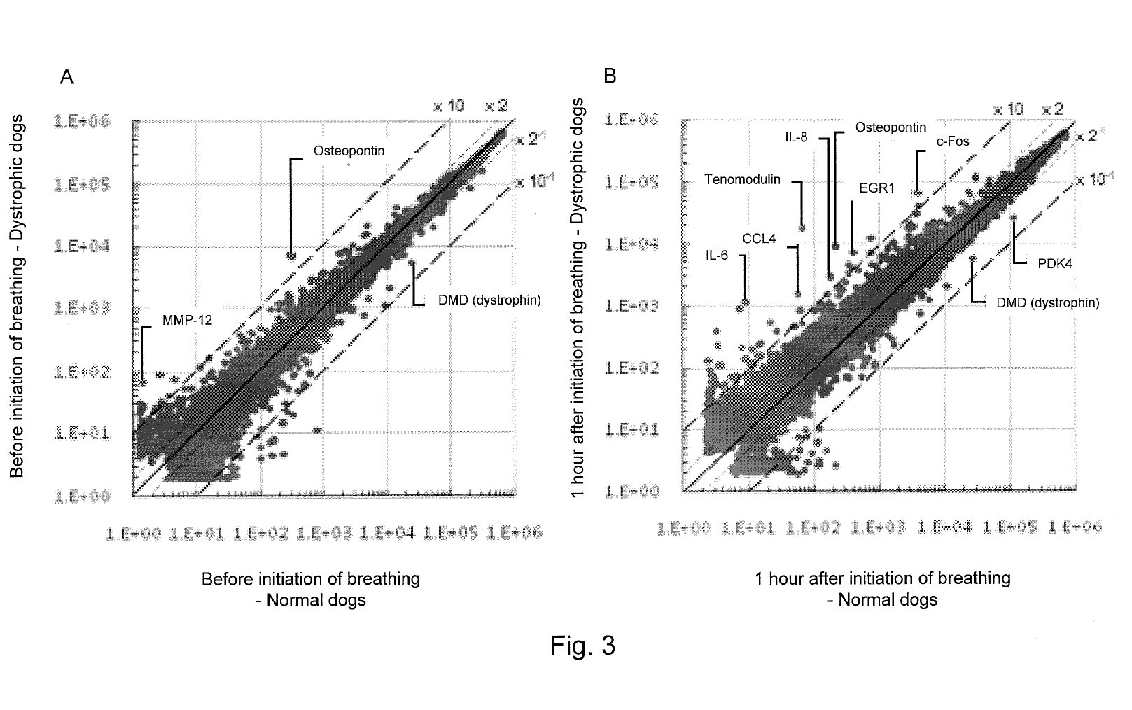

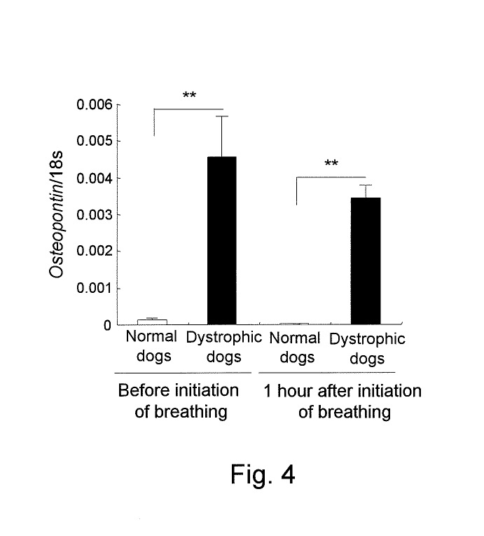

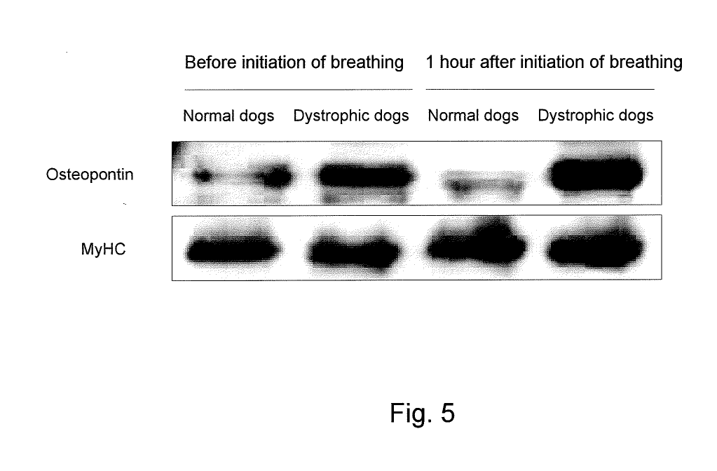

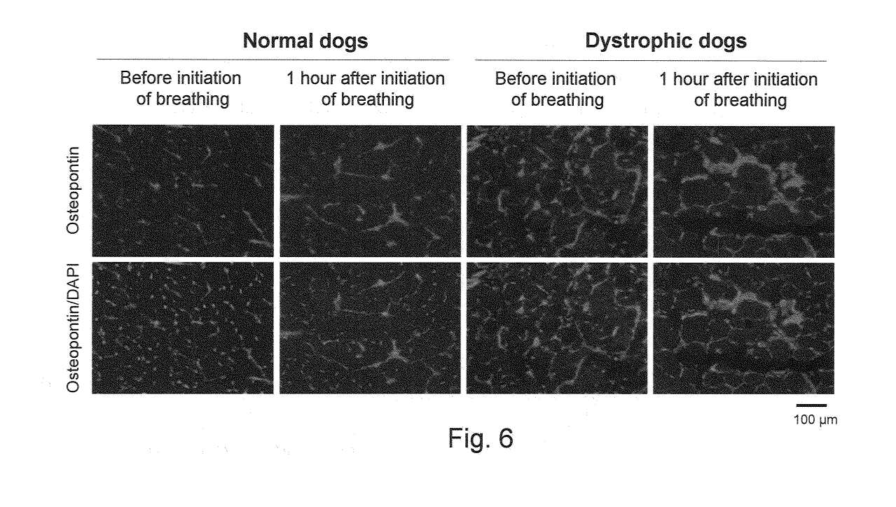

As a result, the osteopontin expression level in the diaphragms of dystrophic dogs before the initiation of breathing was found to be approximately 27 times greater than that of normal dogs, and the elevated expression levels and localization of proteins in the muscle cytoplasm and in the interstitium were confirmed (FIGS. 3 to 6). Also, the osteopontin expression level was high in the diaphragms of dystrophic dogs after the initiation of breathing. Osteopontin is reported to be expressed at high levels at an early stage of regeneration of dystrophin-deficient muscle (Hirata, A, et al., Am. J. Pathol., 2003, 163: 203-205) and to be associated with the acceleration of fibrosis (Vetrone, S. A., et al., J. Clin. Invest., 2009, 119: 1583-1594). Osteopontin is activated by influx of intracellular calcium ions from the stretch-activated channel (Allen, D. G, et al., Can. J. Physiol. Pharmacol., 2010, 88: 83-91), and it functions as a cytokine that induces neutrophils or macrophages (Wang, K. X., et al., Cytokine Growth Fact, 2008, 19: 333-345). If the data by the present inventors and the existing reports are taken into consideration, it is considered that osteopontin may be expressed at a very early stage of muscular dystrophy, may be associated with induction of inflammatory cells, may be associated with fibrosis that occurs at a later stage, and may have an essential role in DMD conditions. At present, osteopontin is considered to be a target molecule for the treatment of DMD (Vetrone, S. A., 2009; and Qureshi, M. M., et al., J. Diet Suppl., 2001, 7: 159-178).

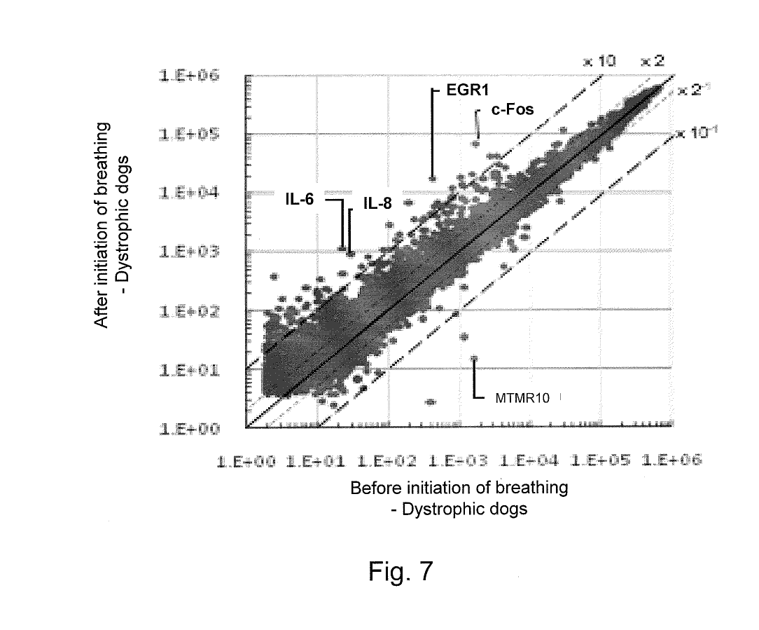

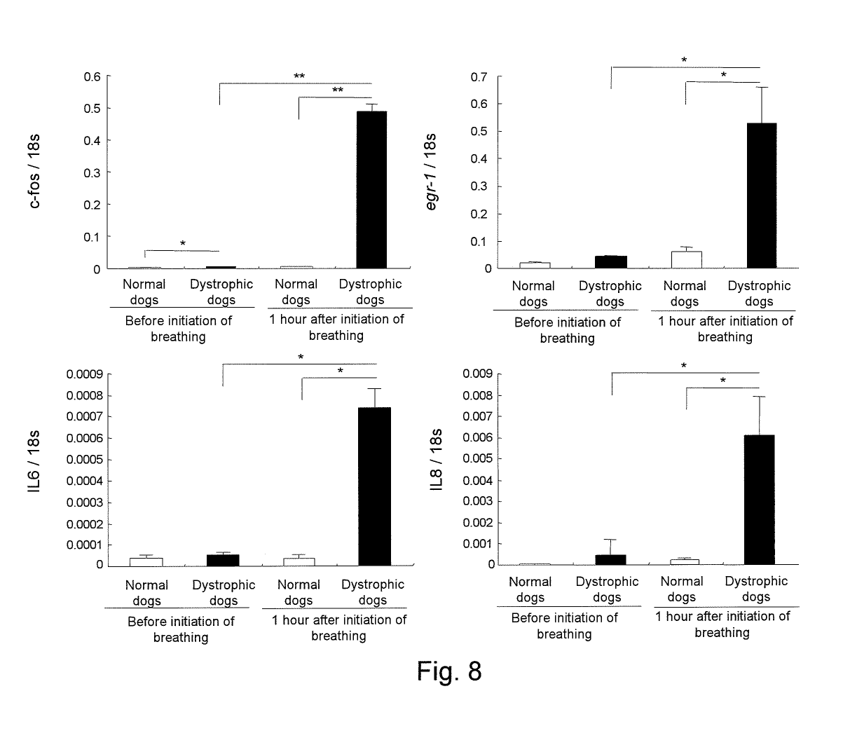

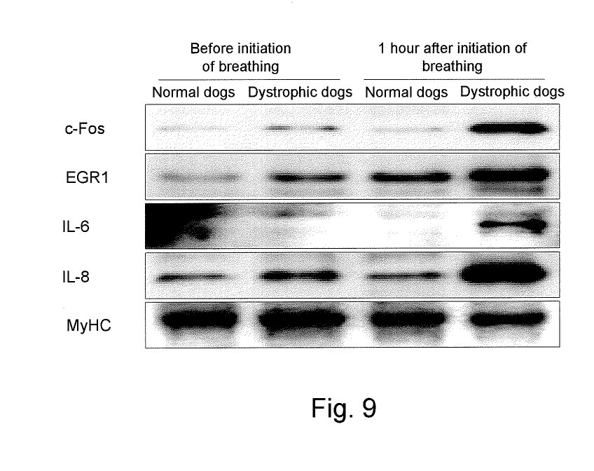

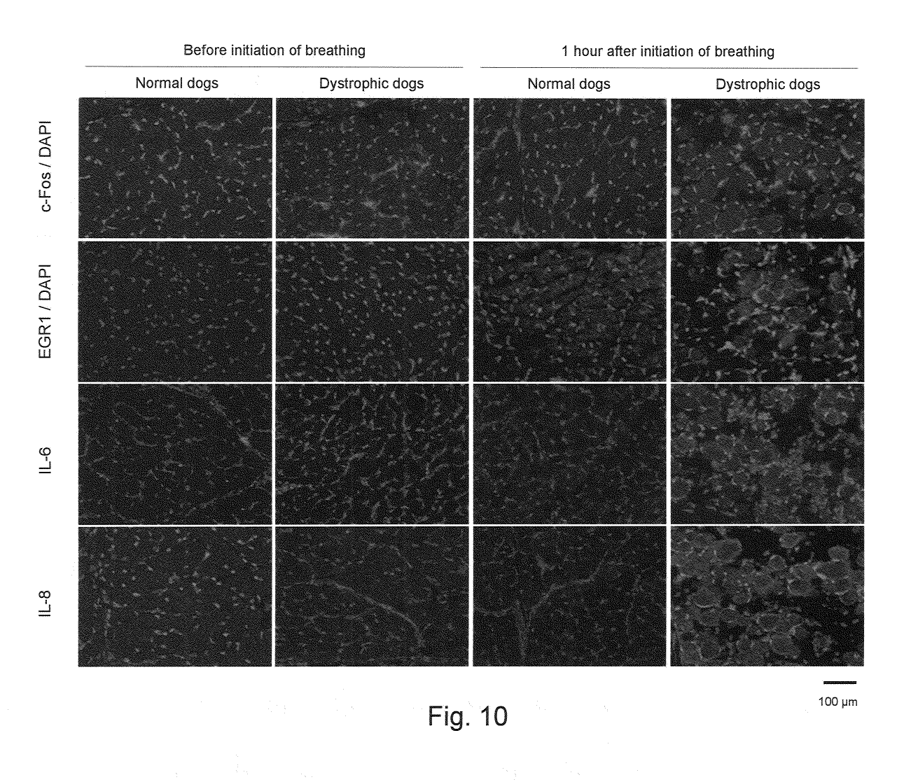

In the diaphragms of dystrophic dogs after the initiation of breathing, potent expression of c-fos and egr-1, which are transcription factors/signaling molecules, was observed in the muscle cell nuclei and cytoplasm (FIGS. 7 to 10). The immediate early genes (i.e., c-fos and egr-1) are regulated by the local concentration of intracellular calcium ions (Schaefer, A, et al., Biochem. J., 1998, 355: 505-511; Grembowicz, K. P., et al., Mol. Biol. Cell, 1999, 10: 1247-1257), and such genes induce various genes located downstream. In addition, the expression levels of the inflammatory/immune response genes (IL-6 and IL-8) were significantly elevated in the diaphragms of dystrophic dogs, and such genes were localized in the muscle cytoplasms (FIGS. 7 to 10). IL-6 and IL-8 are downstream genes of EGR1 (Schuring a, J. J., et al., Cytokine, 2001, 14: 78-87) and c-Fos (Cullen, E. M., et al., Mol. Immunol., 2010, 47: 1701-1709), respectively, and such genes are reported to exhibit elevated expression levels in normal skeletal muscles in the form of cytokines produced in muscle cells (myokines) due to exercises (Pedersen, B. K., et al., J. Appl. Physiol., 2007, 103: 1093-1098). It is pointed out that IL-6 may be associated with the maintenance of the metabolic homeostasis of normal skeletal muscles (Febbraio, M. A., et al., FASEB, J., 2002, 16: 1335-1347); however, it may induce inflammation in dystrophic muscle to which mechanical stress has been applied. IL-8 is a major chemokine that is associated with the induction of neutrophils at a damaged site (Peterson, J. M., et al., J. Appl. Physiol., 2009, 106: 130-137). Based on the fact that the removal of neutrophils with the use of antibodies has resulted in the reduction of myonecrosis in mdx mice, neutrophils are reported to play key roles at an early stage of dystrophy (Hodgetts, S., et al., Neuromuscl Disord., 2006, 16: 591-602). Based on the data presented above, the molecular mechanisms from the application of mechanical stress to invasion of inflammatory cells in dystrophic muscle are considered to have been elucidated.

Thus, the expression levels of c-fos, erg-1, IL-6, and IL-8 were found to be high in dystrophic dogs after the initiation of breathing, and such genes were found to be useful as markers for muscular dystrophy.

All publications, patents, and patent applications cited herein are incorporated herein by reference in their entirety.

1. A method of diagnosing muscular dystrophy in a subject comprising
(a) determining an expression level of at least one marker selected from the group consisting of c-Fos, EGR1, IL-6, and IL-8 in a cell, tissue, or body fluid sample obtained from the subject;
(b) comparing the results of step (a) with a reference value; and
(c) diagnosing muscular dystrophy for the subject by determining that the expression level of the at least one marker is significantly elevated compared with the reference value.

2. The method according to claim 1, wherein the determination of the expression level is conducted by using a DNA primer and/or DNA probe.

3. The method according to claim 1, wherein the determination of the expression level is conducted by using an antibody.

4. The method according to claim 1, wherein the sample is selected from the group consisting of a muscle sample, a blood sample, and a serum sample.

5. The method according to claim 1, wherein diagnosis of muscular dystrophy is evaluation of a muscular dystrophy carrier or prediction of development of muscular dystrophy.

6. A method for screening for a therapeutic agent or a technique for treating muscular dystrophy comprising
(a) treating a muscle cell derived from an animal that had developed muscular dystrophy or a muscular dystrophy carrier animal with the test agent or the technique or combinations thereof;
(b) determining an expression level of at least one marker selected from the group consisting of c-Fos, EGR1, IL-6, and IL-8 in the muscle cell; and
(c) identifying a the test agent or the technique as a candidate for the therapeutic agent or technique for treating muscular dystrophy based on the results obtained in step (b).

7. A method for screening for a therapeutic agent or a technique for treating muscular dystrophy comprising
(a) determining an expression level of at least one marker selected from the group consisting of c-Fos, EGR1, IL-6, and IL-8 in a cell, tissue, or body fluid sample obtained from an animal that had developed muscular dystrophy or a muscular dystrophy carrier animal, which has been treated with the test agent or the technique or combinations thereof; and
(b) identifying the test agent or the technique as a candidate for the therapeutic agent or technique for treating muscular dystrophy based on the results obtained in step (a).

8. The method according to claim 6, which further comprises a step of determining an expression level of at least one marker selected from the group consisting of c-Fos, EGR1, IL-6, and IL-8 in the muscle cell prior to the treatment with the test agent or technique.

9. The method according to claim 6, wherein the test agent or technique is identified as a candidate for the therapeutic agent or technique for muscular dystrophy when the expression level of a marker in the treated muscle cell is lower than that of the same marker in an untreated muscle cell or sample from the same animal.

10. The method according to claim 6, wherein the animal that had developed muscular dystrophy or the muscular dystrophy carrier animal is a human who had developed muscular dystrophy or is a muscular dystrophy carrier, or an animal model of muscular dystrophy.

11. A method for evaluating the efficacy of a therapeutic agent or technique for treating muscular dystrophy comprising:
(a) determining an expression level of at least one marker selected from the group consisting of c-Fos, EGR1, IL-6, and IL-8 in a cell, tissue, or body fluid sample obtained from an animal that had developed muscular dystrophy or a muscular dystrophy carrier animal, which has been treated with a test agent or technique; and
(b) evaluating the efficacy of the test agent or technique based on the results obtained in step (a).

12. The method according to claim 11, wherein the animal that had developed muscular dystrophy or the muscular dystrophy carrier animal is a human who had developed muscular dystrophy or is a muscular dystrophy carrier, or an animal model of muscular dystrophy.

13. The method according to claim 7, which further comprises the step of determining an expression level of at least one marker selected from the group consisting of c-Fos, EGR1, IL-6, and IL-8 in the sample prior to the treatment with the test agent or technique.

14. The method according to claim 7, wherein the test agent or the technique is identified as a candidate for the therapeutic agent or the technique for treating muscular dystrophy when the expression level of a marker in the treated sample is lower than that of the same marker in an untreated muscle cell or sample from the same animal.

15. The method according to claim 7, wherein the animal that had developed muscular dystrophy or the muscular dystrophy carrier animal is a human who had developed muscular dystrophy or is a muscular dystrophy carrier, or an animal model of muscular dystrophy.

16. A method of diagnosing muscular dystrophy in a subject comprising
(a) obtaining a sample of cells, tissue, or body fluid from the subject;
(b) determining an expression level of one or more markers selected from the group consisting of c-Fos, EGR1, IL-6, and IL-8 in the sample;
(c) comparing the expression level of the one or more markers in the sample to the expression level of the one or more markers in a reference sample;
(d) determining that the expression level of the one or more markers in the subject's sample is significantly elevated compared to the expression level of the one or more markers in the reference sample.

17. The method according to claim 16, wherein the reference sample is a sample of tissue or body fluid selected from the group consisting of (1) a subject that does not have muscular dystrophy, (2) a subject that is a known genetic carrier for muscular dystrophy, and (3) a subject that has developed muscular dystrophy.


SEQUENCE LISTING (28 sequences)

SEQ ID NO: 1 - DNA, 6210 bp, Homo sapiens
  CDS (889)..(1029)
  CDS (1783)..(2034)
  CDS (2466)..(2573)
  CDS (2688)..(3326)
gcaggaacagtgctagtattgctcgagcccgagggctggaggttaggggatgaaggtctgcttccacgctttgcactgaattagggctagaattggggatgggggtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttcttcggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacaggtgaggaactctagcgtactcttcctgggaatgtgggggctgggtgggaagcagccccggagatgcaggagcccagtacagaggatgaagccactgatggggctggctgcacatccgtaactgggagccctggctccaagcccattccatcccaactcagactctgagtctcaccctaagaagtactctcatagtttcttccctaagtttcttaccgcatgctttcagactgggctcttctttgttctcttgctgaggatcttattttaaatgcaagtcacacctattctgcaactgcaggtcagaaatggtttcacagtggggtgccaggaagcagggaagctgcaggagccagttctactggggtgggtgaatggaggtgatggcagacacttttactgaatgtcggtctttttttgtgattattctagttatctccagaagaagaagagaaaaggagaatccgaagggaaaggaataagatggctgcagccaaatgccgcaaccggaggagggagctgactgatacactccaagcggtaggtactctgtgggttgctcctttttaaaacttaagggaaagttggagattgagcataagggcccttgagtaagactgtgtcttatgctttcctttatccctctgtatacaggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaaccttgtgctcttttcattctggaagtcttgtaagtttctgaaaggtattattggagaccagtttgtcaagaagggtagctgctggagggggacacaccctctgtctgatcccttatcaaagaggacaaggaaactatagagctgattttagaatattttacaaatacatgccttccattggaatgctaagattttctactgcttctggggacgggaaaccgctgtgtaacagcttttgtgggaatacattttttctgtttcagtactcgcagggggaaatatttaaattttgttgtgctaatattaaattcagatgttttgatcttaaaggaaccctttaagcaaacagaacctagctttgtacagactattttaactttttattctcacaaaatcacgtggagggttattctacttcaaagatgagcaaattgaagaatggttagaataaacaactttcttgatattccgttatcggcattagaatcttcctgctcgttatcgtatccagcaggctgaactgcctcttgatacttggttaaaaaaaattttcaggccgggcgcggtggcccatgcctgtaatcctagcactttgggaggccgaggcaggcggatcacctgaggtcgggagttcgagaccagcctgaccaacatggagaaaccccgtctttactaaaaatacaaaattagcctggtgtggtggtgcatgcctgtaatcctagctacttgagaggctgagacaggaaaatcacttgaactcgggaggcggatgttgcagcgaactgagattgcgccattgcactccagcctgggcaacaagattgaaactctgtttaaaaaaaaaagttttcactaatgtgtacatttttttgtactcttttattctcgaaagggaaggagggctattgccctatcccttattaataaatgcattgtggtttctggtttctctaataccatatgcccttcattcagtttatagtgggcggaagtgggggagaaaaagttgctcagaaatcaaaagatatctcaaacagcacaaataatggctgatcgttctgcaaacaaaaagttacataatagctcaagaaggagaagtcaacatgactctgaacaagctttaacttagaaactttatcatcttaaggaagaacgtgacctttgtccaggacgtctctggtaatggggcacttacacacacatgcacacgtacaaaccacagggaaaggagaccgcccttctgcctctgctcgcgagtatcacgcaggcaccatgcactatgttttcacacacactgggtggaagaagagcttcagcgccagtcttctaatgctttggtgataatgaaaatcactgggtgcttatggggtgtcatattcaatcgagttaaaagttttaattcaaaatgacagttttactgaggttgatgttctcgtctatgatatctctgcccctcccataaaaatggacatttaaaagcaacttaccgctctttagatcactcctatatcacacaccacttggggtgctgtttctgctagacttgtgatgacagtggccttaggatccctgtttgctgttcaaagggcaaatattttatagcctttaaatatacctaaactaaatacagaattaatataactaacaaacacctggtctgaaataacaaggtgatctaccctggaaggaacccagctggtgggccaggagcggtggctcacacctgtaattccagcactttgggaggctgagacaggaggatcactggagtccaggagtttgagaccagcctgggcaacatggcaaaacccagtgtgcttctgttgtcccagctacactactcaggaggctgaggcaggagtatgacttgagcctgggagggggaggttgcagagaactgatattgcaccaccactgcactccagcctgggtgacacagcaaaaccctatctcaaaaaaaaaaaaaaaaaaaaggaacccagctggttcctgtaggtgtgcaataataacaaccagaggaagaaaaggaagacgatttcccagatgaagaagggcagctggaccttcggac

SEQ ID NO: 2 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 3 - DNA, 2102 bp, Canis familiaris
  CDS (156)..(1298)
accgcatctgcagcgagcagccgagaagccgagacggagccggcggccgcggcgcagcgagcgagcagtgaccgcgctcccacccagctctgccccacagctccggcctgtctccgcccctcagcccctcgccccggccctgactcaccgcgaccatgatgttctctggtttcaacgccgactacgaggcgtcctcctcccgctgcagcagcgcgtccccggccggggacaccctctcctactaccactcaccggccgactccttctccagcatgggctctcccgtcaatgcgcaggacttctgcaccgatctggccgtctccagtgccaacttcatcccgacggtgactgccatctccaccagcccggacctgcagtggctggtgcagcccaccctggtctcctccgtggccccgtcccagaccagagccccccacccgtatggagtccccaccccctcggctggggcttactccagggctggcgttgtgaagaccatgacgggaggcagagctcagagcattggccggaggggcaaggtggaacagctgtccccagaagaagaagagaaaaggagaatccgaagggaaaggaataagatggctgcagccaagtgccggaaccgcaggagggagctgactgacacgctccaagcggagacagaccaactagaagacgagaagtctgctctgcagaccgagattgccaacctgctgaaggagaaggagaaactagagttcatcctggcagctcaccgacctgcctgcaagatccctgatgacctgggcttccccgaagagatgtccgtggcttccctagatctgagcgggggcctgcccgaagctgccaccccggagtccgaggaggctttcaccctgcccctcctcaatgatcctgagcccaagccctccgtggagcccgtcaagagcatcggcagcatggagctgaaggccgagccctttgatgacttcctgtttccagcatcatccaggcccagcggctcggagaccgcccgctccgtgccagacatggacctgtctggttccttctatgcagcagactgggagcccctgcatggtggctccctggggatggggcccatggccacagagcccgagcctctgtgcacccccgtagtcacctgtactcctagctgcactacctatacgtcttccttcgtcttcacctaccctgaggctgactccttccccagctgtgcggccgctcatcgcaagggcagcagcagcaacgaaccctcctctgactcgctcagctcacccacgctgctggccctgtgagcaggcagggaggggaggcggcaggcaccctagggtgctactgcccaagttggtgcattacagagaggagaaacacgtcttccctcgagggttcccgtagacctagggaggaccttatctgtgcgcgaaacacaccaggcggtgggcctcaaggacttgaaagcatccacgcgcggcctcaagtccttacctcttccggagatgtagcaaaacgcatggagtgtgtattgttcccagtgacacatctgagagctggtagttagtagcatgttgagccaggcctgggtctgtgtctcttatctctttctctttagtcttctcatagcattaactaatctattgggttcattattggaattaacctggtgctggatattttcgaattgtatctagtgcagctgattttaacaataactactgtgttcccggcaatagtgtgttctgattagcaatgaccaatattaaactaagaaaagatatgactttattttctagtagatagaaataaatagctctatccatgtactgtagttttttcttcaacatcaatgttcattgtaacgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttcaaagacattgtcaataaaagcatttaagttgaatgc

SEQ ID NO: 4 - protein, 380 aa, Canis familiaris
MMFSGFNADYEASSSRCSSASPAGDTLSYYHSPADSFSSMGSPVNAQDFCTDLAVSSANFIPTVTAISTSPDLQWLVQPTLVSSVAPSQTRAPHPYGVPTPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLSGGLPEAATPESEEAFTLPLLNDPEPKPSVEPVKSIGSMELKAEPFDDFLFPASSRPSGSETARSVPDMDLSGSFYAADWEPLHGGSLGMGPMATEPEPLCTPVVTCTPSCTTYTSSFVFTYPEADSFPSCAAAHRKGSSSNEPSSDSLSSPTLLAL

SEQ ID NO: 5 - DNA, 3136 bp, Homo sapiens
  CDS (271)..(1902)
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 6 - protein, 543 aa, Homo sapiens
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 7 - DNA, 3131 bp, Canis familiaris
  CDS (266)..(1894)
aacttggggagccgccgccgccagccgccgccgccgccagcttccgccgccgcaggaccggcccctgccccagccccggtagcagcgccgcgtccgcaccggccggcggcgagggcgagcctggaggccccacctgccctggccacagtgtgccctgcgtcccgcatgtgacccggccaggcccccgagagtgtgtcccccgcagccgcggctccgggctgcgcccacccgccccaacactagctctccagcccgcccgtccgggatggccgcggccaaggccgagatgcagctgatgtctccgctgcagatctccgacccgttcggctcctttcctcactcgcccaccatggacaactatcccaagctggaggagatgatgctgctgagcaacggggctccccagttcctcggtgcagccggggcctcggagggcagcggcggtagcagcagcggcggcagcgggggcggtggaggtggagggggcggcagcggcggcagcagcggcagcgccttcaaccctcagggggaggcgggcgagcagccctacgagcacctgaccgcagagtcttttcccgacatctctctgaataacgagaaggttctggtggagaccagttaccccagccaaaccacgcggctgccgcccatcacctacactggccgcttctctctggagcctgcacccaacagcggcaacaccttgtggccagagcccctcttcagcctggtcagcggcctcgtgagcatgaccaacccaccggccacctcgtcttcggcgccgtctccagcagcctcctcctcctccgccgcctctcagagcccacccctgagctgtgccgtccagtccaacgacagcagccccatttactcggccgcgccgaccttccccacgcctaacagtgacatcttcccggagccgcagagccaggccttcccgggctccacgggcgccgcgctccagtacccgcctcccacctaccctgcggccaagggtggcttccaggtccccatgatccctgactacctgtttccacaacagcagggggacctgggcctgggcacccccgaccagaagcccttccaaggcctggagggccgtacccagcagccttcgctcactccgttgtctaccatcaaggcctttgccacgcagtcgggctcccaggacttgaagaccctcaacaccacttaccagtcccagctcatcaaacccagccgcatgcgcaagtaccccaaccggcccagcaagacgcccccccacgaacgcccgtacgcctgcccggtcgagtcctgcgaccgtcgcttctcccgctccgacgagctcacgcgccacatccgcatccacaccggccagaagcccttccagtgtcgcatctgcatgcgcaacttcagccgcagtgaccatctcaccacccacatccgcacccacacgggcgagaagcccttcgcctgcgacatctgtgggagaaagtttgccaggagcgatgagcgcaagaggcataccaagatccacttaaggcaaaaggacaaaaaagcagacaaaggtgttgtggcctcctcagctgccacctccctctcttcctacccgtcccaggtggctacctcctacacgtccccggttactacctcttatccctccccagccaccacctcctatccgtcacctgtacccacctcctactcctctcccggttcctcaacctacccatcccctgtgcacagtggcttcccctcaccctcagtggccaccacatactcttccgtcccccctgctttcccggcccaagtcagcagcttcccttcctcggctgtcaccaactccttcagcgcctctgcagggctttcggacatgacaaccaccttttctcccaggacaattgaaatctgctgaagggaaaggagaaaccagggaaaagagaaagaaacacaagagacttaagagacaggaggaggagatggccacaggagggggttcctctaggtgagatggaggttctcagagccaaatcctccccctctactccaccccagggctggtgtggaaggtctgttggcctgcgatcctttctgcccacttgcccttcctctgcagttcctactgcctgtgacttcagctgcctgaaacagccatgtccaagttcttcacctctatccaaagaacttgatttgcatggattttggatatatcatttcagtatcatctccatcgtatgcctgacccccccctgctcccttcgatgctagaaaatcaagttggcaaaaatggggtttgggctcctcagagcccagccctgcacccttgtacagtgtctgtgccatggattttgtttttcttggggtactcttgatgtgaagataatttgcatattctattgtattatttggaattaggtcctttggggggggaaaaaaaaaaagaaaagccaagcaaaccaacggtgatcctttattttgtgatgatgctgtgacgattaagtttgaagctttttttgaaacagcagtccttggtattaatcagagcatgtgtcagagtgacgttccgttaactttttgtaaatagtgcccgactgtactctcacacgtgacaaaatatggtttggtttttcttctttttttttgaaagtgttctttttttccgtccttttggtttaaaaagtttcacgtcttggtgccttttgtgtgatgcgccttgctgacagcttgacatgtgcaattgtgagggatgtgctcacctctagccttaaggggggcagggagtgacgattcgggggaggctttgggagcaaaataaggaagagggctgagctaagcctcggctctccagaatgtaagaaaacaaaatttaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttttattttactttgtacttgtgtttgcttaaacaaagtgactgtttggcttataaacacattgaatgcgctttattgcccatgggatatgtggtgtatatccttcagaaaaattaaaaagaaaataaaatagctgcg

SEQ ID NO: 8 - protein, 542 aa, Canis familiaris
MAAAKAEMQLMSPLQISDPFGSFPHSPTMDNYPKLEEMMLLSNGAPQFLGAAGASEGSGGSSSGGSGGGGGGGGGSGGSSGSAFNPQGEAGEQPYEHLTAESFPDISLNNEKVLVETSYPSQTTRLPPITYTGRFSLEPAPNSGNTLWPEPLFSLVSGLVSMTNPPATSSSAPSPAASSSSAASQSPPLSCAVQSNDSSPIYSAAPTFPTPNSDIFPEPQSQAFPGSTGAALQYPPPTYPAAKGGFQVPMIPDYLFPQQQGDLGLGTPDQKPFQGLEGRTQQPSLTPLSTIKAFATQSGSQDLKTLNTTYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKGVVASSAATSLSSYPSQVATSYTSPVTTSYPSPATTSYPSPVPTSYSSPGSSTYPSPVHSGFPSPSVATTYSSVPPAFPAQVSSFPSSAVTNSFSASAGLSDMTTTFSPRTIEIC

SEQ ID NO: 9 - DNA, 1201 bp, Homo sapiens
  CDS (117)..(755)
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 10 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - DNA, 1104 bp, Canis lupus
  CDS (58)..(681)
gccctcgagcccaccaggaacgaaagagagctccatctgccctccaggaccccagctatgaactccctctccacaagcgccttctccctggggctgctcctggtgatggctactgctttccctaccccgggacccctggcaggagattccaaggatgatgccacttcaaatagtctaccactcacctctgcaaacaaagtggaagaactgattaagtacatcctcggcaaaatctctgcactgagaaaggagatgtgtgacaagtttaacaagtgtgaagacagcaaagaggcactggcagaaaataacctacatcttcccaaactggagggaaaagatggatgcttccaatctgggttcaatcaggagacctgcttgacaagaatcactaccggtcttgtggagtttcagctacacctgaatatcctccagaacaactatgagggtgataaggaaaatgtcaagtctgtgcacatgagtaccaagatcctggtccagatgctaaagagcaaggtaaagaatcaggatgaagtgaccactcctgacccaaccacagacgccagcctgcaggctatcttgcagtcgcaggatgagtgcgtgaagcacacaacaattcacctcatcctgcggagtctggaggatttcctgcagttcagtctgagggctgttcggataatgtagcctgggcatctaagattgctgtagttcatgggcattcctttctccagtcagaaacctgtgcagtgggcacaaaacttatgttgttctctgtgaggaactaaaagtatgagcgttaggacactattttaattatttttaatttattgatatttaaatatgtgatatggagttaatttatataagtaatagatatttatattttttatgaagtgccacttgaaatattttatgtattcattttgaaaaagttaacgtaaaatgctatgcggcttgaatatcctcgatgtttcggagccaggtcatttcttggaatgtgtaggtttacctcaaatacatggctaacttatgcatatttttaaaagaaatatttatactgtgtttatataatgtttaaattgtttttataccaataaacaccttttt

SEQ ID NO: 12 - protein, 207 aa, Canis lupus
MNSLSTSAFSLGLLLVMATAFPTPGPLAGDSKDDATSNSLPLTSANKVEELIKYILGKISALRKEMCDKFNKCEDSKEALAENNLHLPKLEGKDGCFQSGFNQETCLTRITTGLVEFQLHLNILQNNYEGDKENVKSVHMSTKILVQMLKSKVKNQDEVTTPDPTTDASLQAILQSQDECVKHTTIHLILRSLEDFLQFSLRAVRIM

SEQ ID NO: 13 - DNA, 1718 bp, Homo sapiens
  CDS (154)..(453)
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 14 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 15 - DNA, 1437 bp, Canis lupus
  CDS (7)..(312)
gtaaacatgacttccaagctggctgttgctctcttggcagcttttgtcctttctgcagctctctgtgaagctgcagttctgtcaagagtcagttcagaacttcgatgccagtgtataaaaacacactccacacctttccatcccaaatatattaaagaactgagagtgattgacagtggcccacattgtgaaaactcagaaatcattgtaaagcttttcaatggaaatgaggtgtgcctggaccccaaggaaaaatgggtacaaaaggttgtgcagatatttctaaagaaggctgagaaacaagatccgtgaaacaacaaacacattctctgtggtttccaagaattcctcaggaaagatgccaatgagacttcaaaaaaatctatttcagtacttcatgtcccgtgtagacctggtgtaggattgccagataaaaatacagtatgcccagttagatttgaatattaagtaaaacaatgaatagtttttttctaaagtctcatatatgttgccctattcaatgtctaggcacacttacattaaacatattattcattgtttgctgtaaattcaaatgtagctggaaatcctggatatattttgttgttgttacatctttccacctcacctacaggccaggatgcatgagtcccttttcaaccttgccttggtctcttctttattcctcaactggagaaaaggtatcagcaagcatcctacctcacagaaatatgaggacatatggaagcactttaactttttctcatgttgtctaaattatgttcaagtgaaacttgtttgcctatttattatttatgtatttatttaagaaacaaatatgggaatatctgtgcataaatttggaaaaataggaaaggaagcattgttgataagttagtataatgatggtagtgaatttatatttattttggtatttagtgatgttatattaaagaactattttgtttttttttttttaaagaactattttgaacaaggttgctagatttagcaaaattaaaaatgagatactcatttaattttgatttcaaacaataattttttattatattattatttatctgaaatttcaattgaaccgcaatcctacttttgatactcctagtcttgtctattcactgacagccttgttcaatgctgggttgaatgatcataaccctgagttagaattgtttctccaaagagcaaaaactcgacaagcaatattaatgaagtaatttcttgccagttaaaatttgtatatttataatatacaaaatagattccttataattttacttattgtgttcttaaacactgacttttttactttaagatgcttttatatgtttcccaagagatttttttttcctcctatttttgatgctatggaaataaaaatgtaaaatatttaaaataaaacttattgtcaaagtc

SEQ ID NO: 16 - protein, 101 aa, Canis lupus
MTSKLAVALLAAFVLSAALCEAAVLSRVSSELRCQCIKTHSTPFHPKYIKELRVIDSGPHCENSEIIVKLFNGNEVCLDPKEKWVQKVVQIFLKKAEKQDP

SEQ ID NO: 17 - DNA, 19 bp, Artificial
  primer
ggaaagtacagccaggtcc

SEQ ID NO: 18 - DNA, 20 bp, Artificial
  primer
acacgaagtccccaaaagtg

SEQ ID NO: 19 - DNA, 20 bp, Artificial
  primer
acgatgtggatagccaggac

SEQ ID NO: 20 - DNA, 20 bp, Artificial
  primer
ggacggcattgaagtcatct

SEQ ID NO: 21 - DNA, 20 bp, Artificial
  primer
actccagggctggcgttgtg

SEQ ID NO: 22 - DNA, 20 bp, Artificial
  primer
agtcagctccctcctgcggt

SEQ ID NO: 23 - DNA, 20 bp, Artificial
  primer
gacaaccaccttttctccca

SEQ ID NO: 24 - DNA, 20 bp, Artificial
  primer
ggcagtaggaactgcagagg

SEQ ID NO: 25 - DNA, 20 bp, Artificial
  primer
gctactgctttccctacccc

SEQ ID NO: 26 - DNA, 20 bp, Artificial
  primer
ttttctgccagtgcctcttt

SEQ ID NO: 27 - DNA, 20 bp, Artificial
  primer
agagtgattgacagtggccc

SEQ ID NO: 28 - DNA, 20 bp, Artificial
  primer
acaccaggtctacacgggac